(19) United States
(12) Patent Application Publication
(10) Pub. No.: US 2022/0098601 A2
(43) Pub. Date: Mar. 31, 2022

(54) METHODS OF REGENERATION AND TRANSFORMATION OF STEVIA PLANT AND TRANSGENIC STEVIA PLANTS HAVING ENHANCED STEVIOL GLYCOSIDES CONTENT

(71) Applicant: TEMASEK LIFE SCIENCES LABORATORY LIMITED, Singapore (SG)

(72) Inventors: In-Cheol JANG, Singapore (SG); Jun-shi ZHENG, Singapore (SG); Mi Jung KIM, Singapore (SG)

(73) Assignee: TEMASEK LIFE SCIENCES LABORATORY LIMITED, Singapore (SG)

(21) Appl. No.: 16/963,133

(22) PCT Filed: Jan. 17, 2019
(86) PCT No.: PCT/SG2019/050028

§ 371 Date: Jul. 17, 2020

Related U.S. Application Data
(60) Provisional application No. 62/619,310, filed on Jan. 19, 2018.
(60) Provisional application No. 62/691,746, filed on Jun. 29, 2018.

(51) Int. Cl. C12N 15/82; A01H 4/00

(52) U.S. Cl. CPC C12N 15/8205; A01H 4/002; A01H 4/008; C12N 15/8243

(57) ABSTRACT

The present invention relates to a method for Agrobacterium-mediated transformation and regeneration of Stevia plants. In particular, the method involves co-culturing leaf explants with Agrobacterium in a medium comprising acetosyringone and 2,4-dichlorophenoxyacetic acid in the dark, callus induction and shoot regeneration in a medium comprising 6-benzylaminopurine, 3-indoleacetic acid, a selective agent and an Agrobacterium eradicant in the dark, and root regeneration in a medium comprising 3-in-doleacetic acid in a light/dark cycle. The present invention also relates to the overexpression of SrDXS I and SrKAH in transgenic plants, resulting in the enhancement of steviol glycosides in the transgenic plants. The present invention further relates to the overexpression SrUGT76G I in transgenic plants, resulting in higher Rebaudioside A (Reb A) to stevioside ratios in the transgenic plants.

### CROSS-REFERENCE TO RELATED APPLICATIONS

The present application is related to and claims priority to U.S. patent application Ser. No. 62/691,746 filed 29 Jun. 2018 and U.S. patent application Ser. No. 62/619,310 filed 19 Jan. 2018. Each application is incorporated herein by reference in its entirety.

### SEQUENCE LISTING

The present application is being filed along with a Sequence Listing in electronic format. The Sequence Listing is entitled 2577259PCTSequenceListing.txt, created on 11 Jan. 2019 and is 73 kb in size. The information in the electronic format of the Sequence Listing is incorporated herein by reference in its entirety.

### BACKGROUND OF THE INVENTION

The present invention relates to the field of plant biotechnology. More specifically, the present invention relates to the regeneration and transformation of Stevia, such as Stevia rebaudiana, plants. The present invention also relates to the overexpression SrDXS1 and SrKAH in transgenic plants resulting in the enhancement of steviol glycosides in the transgenic plants. The present invention further relates to the overexpression SrUGT76G1 in transgenic plants resulting in higher Rebaudioside A (Reb A) to stevioside ratios in the transgenic plants.

The publications and other materials used herein to illuminate the background of the invention or provide additional details respecting the practice, are incorporated by reference, and for convenience are respectively grouped in the Bibliography.

Stevia rebaudiana is a perennial shrub that belongs to the Asteraceae family. It produces steviol glycosides (SGs) that range from 150 to 300 times as sweet as sucrose, making it unique among plants (Ceunen et al., 2013). SGs are mainly accumulated in the leaves of Stevia, accounting for around 4-20% of their dry weight (Lemus-Mondaca et al., 2012). In general, stevioside is the predominant SG present followed by rebaudioside A (Reb A) and Reb C. Dulcoside A, Reb F, steviolbioside, Reb D and Reb E are also frequently detected. By also taking into account the SGs that are only found in trace amounts from certain cultivars of Stevia, a total of more than 30 SGs are currently known to be produced in Stevia (Ceunen and Geuns, 2013). In Paraguay where Stevia is native to, people have long been using it to sweeten their teas and medicine (Kinghorn, 2003). In recent times, the value of Stevia leaf extracts or specific SGs, like Rebaudioside A (Reb A) and Reb D, as a zero calorie natural sweetener has also gained recognition beyond its native country, leading to the introduction of Stevia as a commercial crop in many other countries (Ceunen et al., 2013).

SGs are a group of diterpenoids with varying levels of sweetness depending on the different number and types of sugar moieties (glucose, rhamnose, or xylose) substituted on its aglycone, steviol (Tanaka, 1997). Steviol is synthesized through the methylerythritol phosphate (MEP) pathway in the chloroplast (Totté et al., 2000). The first step in the MEP pathway involves the condensation of pyruvate and d-glyceraldehyde-3-phosphate into 1-deoxy-d-xylulose-5-phosphate (DXP) by DXP synthase (DXS; Rodriguez-Concepcion and Boronat, 2002). After six more steps of conversion, the final enzyme 4-hydroxy-3-methylbut-2-enyl pyrophosphate reductase converts (E)-4-hydroxy-3-methylbut-2-enyl pyrophosphate into isopentenyl pyrophosphate (IPP) and dimethylallyl pyrophosphate (DMAPP), which are the basic five-carbon precursors for the formation of all terpenoids. For the production of SGs and other diterpenoids, two intermediates, IPP and DMAPP, undergo consecutive condensation to form C₂₀ geranylgeranyl pyrophosphate (GGPP). GGPP is then further cyclized to (−)-kaurene and subsequently oxidized to kaurenoic acid (Humphrey et al., 2006; Richman et al., 1999). All steps leading to the formation of kaurenoic acid are also common to gibberellic acid (GA) biosynthesis (Brandle and Telmer, 2007). However, the hydroxylation of kaurenoic acid at C-13 position by kaurenoic acid hydroxylase (KAH) diverts it towards SG biosynthesis (Brandle and Telmer, 2007). Finally, UDP-glycosyltransferases (UGTs) add sugar moieties at the C-13 or C-19 position of steviol to produce a variety of SGs (Richman et al., 2005).

SGs are synthesized from the glycosylation of steviol aglycone, which is derived from the methylerythritol phosphate (MEP) pathway. Each SGs have different number and combination of sugar moieties attached at the C₁₉ or the C₁₃ position of steviol (Ceunen and Geuns, 2013). All SGs contain β-D-glucose as their common sugar moiety but some SGs such as Reb C, Reb F and dulcoside A also have rhamnose and xylose added along with glucose. The addition of the activated sugars to aglycone acceptors are carried out by UDP-glycosyltransferase (UGTs) (Richman et al., 2005). UGTs are considered to be promiscuous but they exhibit regioselectivity in the substrates they convert (Hansen et al., 2003). For the biosynthesis of SGs, four UGTs, SrUGT74G1, SrUGT76G1, SrUGT85C2 and SrUGT91D2, have been identified in Stevia so far. These Stevia UGTs contain the highly conserved plant secondary product glycosyltransferase (PSPG) motif of plant-derived family 1 UGTs on their C-terminus (Gachon et al., 2005; Richman et al., 2005). Each of them catalyzes the addition of a sugar moiety at specific positions. SrUGT85C2 and SrUGT74G1 are known to glucosylate the C₁₃ hydroxyl position and the C₁₉ carboxylic acid position of the steviol aglycone, respectively (Richman et al., 2005). On the other hand, SrUGT91D2 is able to further glucosylate the glucose attached on either the C₁₃ or C₁₉ position to form a 1,2-β-D-glucosidic linkage (1,2-β-D-glucosylation) in the absence of a 1,3-glucose (Olsson et al., 2016). For SrUGT76G1, it catalyzes the glucosylation of the glucose moieties as well but forms a 1,3-β-D-glucosidic linkage (1,3-β-D-glucosylation) instead, and the presence of a 1,2-glucose at SGs does not affect its activity (Richman et al., 2005; Olsson et al., 2016).

Although SGs are generally sweet, organoleptic properties of individual SGs depend on the combination of sugar moieties attached to steviol (Hellfritsch et al., 2012). Therefore, other than increasing overall SGs content, there is also a preference for Stevia varieties that can produce the more pleasant tasting SGs in greater proportions. Comparing between the two most abundant SGs in Stevia, Reb A is sweeter and less bitter tasting than stevioside and is thus more valuable as a sweetener (Singla and Jaitak, 2016). In the SGs biosynthesis pathway, stevioside can be converted to Reb A by SrUGT76G1 (Richman et al., 2005). Furthermore, SrUGT76G1 is also involved in the biosynthesis of Reb M, which has a more superior taste profile than Reb A but has only been detected in trace amounts in certain Stevia cultivars (Prakash et al., 2014; Olsson et al., 2016).

For increasing the levels of specific glycosylated metabolites, overexpression of the UGTs involved has been shown to be a feasible approach in plants. In Rhodiola sachalinensis, which is well-known for the production of salidroside, the overexpression of RsUGT73B6 led to an increase in salidroside content (Ma et al., 2007). Additionally, overexpression of AtUGT73C6 and AtUGT71C5 in Arabidopsis has also been demonstrated to increase brassinosteroid glucoside and abscisic acid-glucose ester, respectively (Husar et al., 2011; Liu et al., 2015). Therefore, the overexpression of Stevia UGTs in Stevia may increase total SGs content or promote the synthesis of preferred SGs.

Many Stevia genes uncovered from the next-generation sequencing are now publicly available (Chen et al., 2014; Kim et al., 2015). However, a reliable Stevia transformation technology remains to be developed for the functional genomics of Stevia and the generation of new Stevia with improved traits such as greater sweetness and resistance towards pest and diseases. Although Agrobacterium-mediated Stevia transformation using β-glucuronidase (GUS) reporter gene was introduced (Khan et al., 2014), no further transgenic Stevia has been reported so far, which may result from the absence of a reliable transformation method. Tobacco plants have been routinely transformed using Agrobacterium and its protocol could be conveniently adapted to plants of Solanaceae family (Bevan et al., 1983; Horsch et al., 1985; van der Meer, 2006; Yin et al., 2017). However, transformation of other important crops such as soybean and corn required further optimization of their specific regeneration strategies (Ganeshan et al., 2002). For Stevia, although there are a few protocols describing shoot regeneration from leaf explants, there has been a lack of consensus on the conditions used (Aman et al., 2013; Anbazhagan et al., 2010; Das and Mandal, 2010; Khalil et al., 2014; Patel and Shah, 2009). Therefore, the development of a new and efficient method for regeneration and genetic transformation of Stevia would be required for a broad range of biotechnological applications as well as functional genomic studies of Stevia.

It is desired to develop an efficient and reliable method for the regeneration of Stevia and for the Agrobacterium-mediated transformation of Stevia. It is also desired to produce transgenic Stevia plants that have modulated expression of one or more genes in the MEP pathway for enhanced content of SGs. It is also desired to produce transgenic Stevia plants that have modulated expression of one or more genes for enhanced content of rebaudiosides.

### SUMMARY OF THE INVENTION

The present invention relates to the field of plant biotechnology. More specifically, the present invention relates to the regeneration and transformation of Stevia, such as Stevia rebaudiana, plants. The present invention also relates to the overexpression SrDXS1 and SrKAH in transgenic plants resulting in the enhancement of steviol glycosides in the transgenic plants. The present invention further relates to the overexpression SrUGT76G1 in transgenic plants resulting in higher Rebaudioside A (Reb A) to stevioside ratios in the transgenic plants.

Thus, in one aspect, the present invention provides an efficient and reproducible method for regeneration of Stevia. In some embodiments, explants are obtained from the second and third leaves from in vitro propagated Stevia plants. In some embodiments, the explants are cultured on callus induction medium (CIM) which comprises MS mineral salts, MS vitamins, sucrose and 6-benzylaminopurine (BA) and 3-indoleeacetic acid (IAA) as plant hormones for a period of time for the formation of callus. In some embodiments, callus tissue is then transferred to a shoot induction medium (SIM) which comprises MS mineral salts, MS vitamins, sucrose and BA and IAA as plant hormones for a period of time for the formation of shoots. In some embodiments, the shoots are transferred to a rooting medium (RM) which comprises MS mineral salts, MS vitamins, sucrose and IAA as a plant hormone. In some embodiments, after rooting, the plantlets are transferred to potting soil mixed with sand. In some embodiments, the explants are first cultured in a co-culturing medium (CCM) which comprises MS mineral salts, MS vitamins, sucrose and 2,4-dichlorophenoxyacetic acid (2,4-D) as a plant hormone prior to culturing on the CIM. In some embodiments, the CCM further comprises acetosyringone. In some embodiments, the culturing on CCM, CIM and SIM are done in the dark. In some embodiments, the CIM, SIM and RM are solid media. In some embodiments, the Stevia plant is a Stevia rebaudiana plant. In some embodiments, the Stevia rebaudiana plant is a Stevia rebaudiana Bertoni plant.

In some embodiments, the Stevia plants and regenerated Stevia plants are propagated and maintained in vitro by cutting and transferring apicals onto RM every few weeks. In some embodiments, the in vitro plants are kept in a Light/Dark (LD) (16 h L/8 h D) plant growth chamber maintained at about 25° C. In some embodiments, after rooting, in vitro plants were transferred to potting soil mixed with sand and covered with a transparent plastic dome for hardening.

In another aspect, the present invention provides an efficient and reproducible method for Agrobacterium-mediated transformation of Stevia plants. In some embodiments, the Agrobacterium-mediated transformation of Stevia plants utilizes the same basic scheme as described above for the regeneration of Stevia plants. In some embodiments for transformation, the explants are first co-cultured with Agrobacterium cells in CCM prior to transfer to the CIM with subsequent transfers to the SIM and RM as described above. In some embodiments, the CCM described above for regeneration further comprises acetosyringone when used for culturing the Stevia plant explants and the Agrobacterium cells. In some embodiments, the CIM described above for regeneration further comprises a selective agent and an Agrobacterium eradicant. In some embodiments, the SIM described above for regeneration further comprises a selective agent and an Agrobacterium eradicant. In some embodiments, conventional selective agents and conventional Agrobacterium eradicants can be used for the Agrobacterium-mediated transformation of Stevia plants. In some embodiments, the culturing on CCM, CIM and SIM are done in the dark. In some embodiments, the CIM, SIM and RM are solid media. In some embodiments, the Stevia plant is a Stevia rebaudiana plant. In some embodiments, the Stevia rebaudiana plant is a Stevia rebaudiana Bertoni plant.

In some embodiments, the transgenic Stevia plants are propagated and maintained in vitro by cutting and transferring apicals onto RM every few weeks. In some embodiments, the in vitro transgenic plants are kept in a Light/Dark (LD) (16 h L/8 h D) plant growth chamber maintained at about 25° C. In some embodiments, after rooting, in vitro transgenic plants are transferred to potting soil mixed with sand and covered with a transparent plastic dome for hardening.

In a further aspect, the present invention provides transgenic Stevia plants having an enhanced content of steviol glycosides. In some embodiments, transgenic Stevia plants are prepared in accordance with the transformation method described herein to overexpress the Stevia 1-deoxy-d-xylulose-5-phosphate (DXP) synthase 1 (DXS1). In some embodiments, transgenic Stevia plants are prepared in accordance with the transformation method described herein to overexpress Stevia ent-kaurenoic acid 13-hydroxylase (KAH). In some embodiments, Stevia DXS1 and KAH are Stevia rebaudiana DXS1 (SrDXS1) and Stevia rebaudiana KAH (SrKAH), respectively. In some embodiments, DXS1 and KAH are stably integrated into the genome of the transgenic Stevia plants. Transgenic Stevia plants are maintained and propagated as described herein.

In an additional aspect, the present invention provides transgenic Stevia plants having an enhanced content of rebaudiosides. In some embodiments, transgenic Stevia plants are prepared in accordance with the transformation method described herein to overexpress the Stevia UDP-glycosyltransferase 76G1 (UGT76G1). In some embodiments, Stevia UGT76G1 is Stevia rebaudiana UGT76G1 (SrUGT76G1). In some embodiments, UGT76G1 is stably integrated into the genome of the transgenic Stevia plants. Transgenic Stevia plants are maintained and propagated as described herein.

### BRIEF DESCRIPTION OF THE FIGURES

FIGS. 1a-1h show Agrobacterium-mediated transformation of Stevia using Condition F. FIG. 1a: The red arrows indicate the second and third leaves that were used as the explant source. FIG. 1b: Leaf explants on CCM. FIG. 1c: Induced callus on CIM. FIG. 1d: Transformed callus showing GFP fluorescence under a fluorescence stereomicroscope. FIG. 1e: Shoots regenerated from calli on SIM. FIG. 1f: Shoot regenerated from transformed calli showing GFP fluorescence under a fluorescence stereomicroscope. FIG. 1g: Regenerated shoots on RM. FIG. 1h: Rooting of regenerated shoots on RM. Scale bars=1 cm for FIGS. 1a-1c, 1e, 1g and 1h; 1 mm for FIGS. 1d and 1f. CCM, co-cultivation media; CIM, callus induction media; SIM, shoot induction media; RM, rooting media.

FIGS. 2a-2c show representative phenotypes of callus on callus induction media. FIG. 2a: Calli induced on media containing 1 mg/L BA and 1 mg/L NAA after 6 weeks. FIG. 2b: Calli and shoot regenerated on media containing 1 mg/L BA and 1 mg/L IAA after 6 weeks. FIG. 2c: Leaf explants placed for one month on media with 1 mg/L BA and 1 mg/L IAA either under 16 h L/8 h D photoperiod (upper panel) or under continuous darkness (lower panel). Scale bar=1 cm

FIGS. 3a and 3b show representative phenotypes of the regenerated shoots. FIG. 3a: Unhealthy looking regenerated shoots with watery and translucent appearance and slight browning. FIG. 3b: Healthy looking callus with few shoots typical of regenerated shoots under Condition E. Scale bar=0.5 cm.

FIGS. 4a and 4b shows characterization of SrDXSs. FIG. 4a: Complementation assay of Stevia DXSs using E. coli DXS deficient mutant (dxs⁻). Transformed cells were grown on LB plates containing either with 0.5 mM mevalonate (+MVA) or without mevalonate (−MVA). E. coli dxs⁻ with pDEST17 (empty vector) and AtDXS1 served as negative and positive controls, respectively. FIG. 4b: Subcellular localization of SrDXS1. Auto, chlorophyll autofluorescence; YFP, YFP channel image; Light, light microscope image; Merged, merged image between Auto and YFP channels. Scale bar=10 μm.

FIGS. 5a-5c show identification of transgenic Stevia plants overexpressing SrDXS1 (SrDXS1-OE) or SrKAH (SrKAH-OE). FIG. 5a: Schematic maps of T-DNA region of pK7WG2D-SrDXS1 and pK7WG2D-SrKAH used for Stevia transformation. LB, left border; nptII, neomycin phosphotransferase marker gene under the terminator and promoter of nopaline synthase gene; T35S and P35S, terminator and promoter of the cauliflower mosaic virus gene respectively; attB2 and attB1, gene recombination sites; SrDXS1, Stevia 1-deoxy-d-xylulose-5-phosphate synthase 1; SrKAH, Stevia kaurenoic acid hydroxylase gene; EgfpER, enhanced green-fluorescent protein gene fused to endoplasmic reticulum targeting signal; ProID, rol root loci D promoter; XbaI and HindIII, sites digested by XbaI and HindIII, respectively, for Southern blot analysis; Probe, probe used for Southern blot analysis. FIG. 5b: Images of GFP signals from leaves and roots of representative SrDXS1-OE #6 or SrKAH-OE #4 under a fluorescence stereomicroscope. WT, wild type. Scale bar=1 mm. FIG. 5c: Confocal images of the leaf underside and roots of WT, representative SrDXS1-OE #6 or SrKAH-OE #4. Auto, chlorophyll autofluorescence; GFP, GFP channel image; Light, light microscope image; Merged, merged image between Auto and GFP channels. Scale bar=5 μm.

FIG. 6 shows confirmation of GFP presence in transgenic lines by immunoblot analysis. Total leaf protein was extracted from, SrDXS1-OE, SrKAH-OE and WT lines and probed with α-GFP antibody. Lower panel shows blot after staining with coomassie blue.

FIGS. 7a-7d shows genomic analysis of transgenic Stevia plants overexpressing SrDXS1 (SrDXS1-OE) or SrKAH (SrKAH-OE). FIGS. 7a and 7b: SrDXS1 (FIG. 7a) or SrKAH (FIG. 7b) amplified from the gDNA of each transgenic Stevia lines. M1, 2-Log DNA ladder. PC, positive control amplified from the respective vector constructs. FIGS. 7c and 7d: Southern blot analysis of SrDXS1-OE (FIG. 7c) or SrKAH-OE lines (FIG. 7d). WT, wild type. M2, DIG-labelled DNA molecular weight marker II.

FIGS. 8a and 8b show expression analysis of SrDXS1 or SrKAH in transgenic Stevia plants. FIGS. 8a and 8b: Relative fold change in SrDXS1 (FIG. 8a) and SrKAH (FIG. 8b) transcript levels among the transgenic Stevia lines overexpressing SrDXS1 (SrDXS1-OE) and SrKAH (SrKAH-OE), respectively. Expression levels of both genes were normalized to that of actin and compared to that of wild type (WT). The values are expressed as mean±SE (n=3). Student's t-test was used for the analysis of statistical significance (*: p<0.05, **: p<0.01)

FIGS. 9a and 9b show representative chromatograms from UHPLC analysis of Steviol glycosides. FIG. 9a: Chromatogram of leaf extract from SrDXS-OE #5 compared to that of the Wild type (WT) and standard sample mixture (Standard) of nine steviol glycosides (Rebaudioside D, Rebaudioside A, Stevioside, Rebaudioside F, Rebaudioside C, Dulcoside A, Rubusoside, Rebaudioside B, Steviolbioside) as indicated on the diagram. FIG. 9b: Chromatogram of leaf extract from SrKAH-OE #1 aligned with that of WT and Standard

FIGS. 10a-10f show analysis of steviol glycosides (SGs) content in transgenic Stevia plants. FIGS. 10a-10f: Total SGs (FIGS. 10a and 10d), stevioside (FIGS. 10b and 10e) and Reb A (FIGS. 10c and 10f) content in the transgenic Stevia lines overexpressing either SrDXS1 (SrDXS1-OE) or SrKAH (SrKAH-OE). Data are presented as mean±SE. Statistical analysis was carried out using Student's t-test relative to wild type (WT) (n=5, *: p<0.05, **: p<0.01).

FIGS. 11a and 11b show relative content of Reb C and Dulcoside A detected from the dried leaves of transgenic Stevia. FIG. 11a: Amount of Reb C and Dulcoside A relative to the vector-only control lines in the SrDXS1 overexpressing lines (SrDXS1-OE). FIG. 11b: Relative abundance of Reb C and Dulcoside A in the SrKAH overexpression lines (SrKAH-OE) relative to the wild type (WT) control lines. All SGs were detected via HPLC at wavelength of 210 nm. Statistical analysis were carried out using Student's t-test (n=5, *p<0.05, ** p<0.01). Data are presented as mean±SE.

FIGS. 12a-12f show phenotypic analysis of transgenic Stevia plants. FIGS. 12a and 12b) Representative transgenic Stevia plants overexpressing SrDXS1 (SrDXS1-OE) (FIG. 12a) or SrKAH (SrKAH-OE) (FIG. 12b) one week after hardening in the soil. FIGS. 12c and 12d: Representative leaf harvested from third node position of SrDXS1-OE lines (FIG. 12c) or SrKAH-OE lines (FIG. 12d) one month after being transferred to the soil. FIGS. 12e and 12f: Average length of the third and fourth internodes in the SrDXS1-OE (FIG. 12e) or SrKAH-OE (FIG. 12f) lines one month after being transferred to the soil. All measurements were expressed as mean±SE (n=5). Wild type (WT) and vector-only line were included as a control. Scale bar=1 cm

FIGS. 13a-13c show analysis of other metabolites derived from MEP pathway. FIGS. 13a and 13b: Relative chlorophylls content and total carotenoids content in the transgenic Stevia plants overexpressing SrDXS1 (SrDXS1-OE) (FIG. 13a) or SrKAH (SrKAH-OE) (FIG. 13b). FIG. 13c: The relative amount of the monoterpenes, α-pinene, β-pinene, and linalool, extracted from leaves of the SrDXS1-OE lines. All measurements were expressed as mean±SE and statistical analysis was carried out using Student's t-test (n=5).

FIGS. 14a-14e show a molecular analysis of transgenic Stevia plants. FIG. 14a: Schematic representation of T-DNA region of the Stevia transformation construct (pK7WG2D-SrUGT76G1). RB and LB, right and left border; rolD, rol root loci D promoter; EGFP-ER, enhanced green-fluorescent protein gene fused to endoplasmic reticulum targeting signal; T35S and CaMV35S, terminator and promoter of cauliflower mosaic virus 35S gene; nptII, neomycin phosphotransferase marker gene; HindIII, Enzyme site used for Southern blot analysis. Arrows indicate primers for genomic DNA PCR. FIG. 14b: Images of GFP signal from leaves and roots of SrUGT76G1-OE lines under a fluorescence stereomicroscope. WT, wild type. Scale bar=1 mm. FIG. 14c: Genomic DNA (gDNA) PCR amplification of SrUGT76G1 from the gDNA of each transgenic lines using forward primer specific to 35S promoter region and reverse primer specific to the 3′ end of SrUGT76G1. FIG. 14d: Southern blot analysis showing transgene copy number. gDNA from each line were digested with HindIII and probed with DIG-labeled probe specific for full-length of CaMV 35S promoter. FIG. 14e: Transcript levels of SrUGT76G1 in SrUGT76G1-OE lines. The relative fold change in SrUGT76G1 expression level among the transgenic lines were normalized to that of WT and expressed as mean±SE (n=3). Ml; 2-Log DNA ladder, M2; DIG-labeled DNA Molecular Weight Marker II-Lambda HindIII-digested marker.

FIG. 15 shows HPLC chromatogram showing steviol glycosides (SGs) content from four UGT76G1-OE lines. Individual SGs are identified by their alignment with the retention time of authentic standards. WT, wild type.

FIGS. 16a-16d show an analysis of steviol glycosides (SGs) content in SrUGT76G1-OE lines. FIG. 16a: The total concentration of SGs derived from the sum of the top four SGs (stevioside, Reb A, Reb C, dulcoside A). FIGS. 16b and 16c: Concentration of stevioside (FIG. 16b) and Reb A (FIG. 16c) from dried leaves of SrUGT76G1-OE lines and wild type (WT). FIG. 16d: Ratio of Reb A to stevioside in SrUGT76G1-OE lines and WT. All SGs detected by HPLC were expressed as a percentage of their dry weight (% w/w DW) with mean±SE. Statistical analysis were carried out using student's t-test relative to WT plants (n=5, *p<0.05, **p<0.01, and ***p<0.001).

FIGS. 17a and 17b show an analysis of steviol glycosides (SGs) content in SrUGT76G1 transgenic lines. Analysis of steviol glycosides extracted from dried leaves of SrUGT76G1-OE lines and WT. FIGS. 17a and 17b: Concentration of dulcoside A (FIG. 17a) and Reb C (FIG. 17b) in each line relative to concentration in WT. All SGs were detected via HPLC. Standard errors were represented by the error bars. Statistical analysis were carried out using student's t-test relative to WT plants (n=5, *p<0.05, **p<0.01)

FIGS. 18a-18i show a phenotypic analysis of transgenic Stevia plants. FIG. 18a: Upper panel, representative whole transgenic Stevia plants overexpressing SrUGT76G1 (SrUGT76G1-OE). Lower panel, leaf harvested from third node position of two-month old SrUGT76G1-OE lines. FIGS. 18b and 18c: Average length (FIG. 18b) and thickness (FIG. 18c) of the third and fourth internodes from two-month old SrUGT76G1-OE lines. FIGS. 18d and 18e: Average length (FIG. 18d) and width (FIG. 18e) of leaves from the third node. FIGS. 18f-18h: Relative contents of chlorophyll a (FIG. 18f), chlorophyll b (FIG. 18g), and total carotenoids (FIG. 18h) in leaves from SrUGT76G1-OE transgenic lines. FIG. 18i: Ratio of chlorophyll a to b. All measurements were expressed as mean±SE (n=5). WT, wild type. Scale bar=10 mm.

FIGS. 19a-19c show transcript levels of genes in the SGs biosynthesis pathway in SrUGT76G1-OE lines. FIGS. 19a-19c: Transcript levels of genes involved in the methylerythritol phosphate (MEP) pathway (FIG. 19a), isoprenoid biosynthesis (FIG. 19b) and the glycosylation of steviol (FIG. 19c). All measurements were expressed as mean±SE (n=5). WT, wild type. SrDXS1, 1-deoxy-D-xylulose 5-phosphate synthase 1; SrDXR1, 1-deoxy-D-xylulose 5-phosphate reductoisomerase; SrCMS, 4-(cytidine 5′ diphospho)-2-C-methyl-D-erythritol synthase; SrCMK, 4-(cytidine 5′ diphospho)-2-C-methyl-D-erythritol kinase; SrMCS, 2-C-methyl-D-erythritol 2,4-cyclodiphosphate synthase; SrHDS, (E)-4-hydroxy-3-methylbut-2-enyl diphosphate synthase; SrHDR, (E)-4-hydroxy-3-methylbut-2-enyl diphosphate reductase; SrGGDPS3, Geranylgeranyl diphosphate synthase 3; SrCPS, Copalyl pyrophosphate synthase; SrKS1, Kaurene synthase 1; SrKO1, Kaurene oxidase 1; SrKAH, Kaurenoic acid hydroxylase.

FIGS. 20a-20c show SrUGT76G1 activity assay using dulcoside A as the substrate. FIGS. 20a and 20b: Chromatograms from TLC (FIG. 20a) and HPLC (FIG. 20b) after reaction between dulcoside A and GST-UGT76G1 or GST-only. Standards, 11 SGs authentic standards. FIG. 20c: Proposed schematic glycosylation reaction performed by SrUGT76G1 on dulcoside A. St, stevioside; Dul A, dulcoside A; Glc, glucose; Rha, rhamnose; 1, Reb E; 2, Reb D; 3, Reb M; 4, Reb I; 5, Reb A; 6, Stevioside; 7, Reb F; 8, Reb C; 9, Dulcoside A; 10, Rubusoside; 11, Reb B.

FIG. 21 shows an in vitro assay of GST-protein activity. HPLC chromatograms of products from assay containing GST or GST-SrUGT76G1 only without substrate as a negative control

### DETAILED DESCRIPTION OF THE INVENTION

The present invention relates to the field of plant biotechnology. More specifically, the present invention relates to the regeneration and transformation of Stevia, such as Stevia rebaudiana, plants. The present invention also relates to the overexpression SrDXS1 and SrKAH in transgenic plants resulting in the enhancement of steviol glycosides in the transgenic plants. The present invention further relates to the overexpression SrUGT76G1 in transgenic plants resulting in higher Rebaudioside A (Reb A) to stevioside ratios in the transgenic plants.

Stevia rebaudiana produces sweet-tasting steviol glycosides (SGs) in its leaves which can be used as natural sweeteners. Metabolic engineering of Stevia offers an alternative approach to conventional breeding for the enhancement of SGs production. However, an effective protocol for Stevia transformation has been lacking in the art. An efficient and reproducible method for in vitro shoot regeneration and Agrobacterium-mediated transformation of Stevia described herein. As described herein, it has been discovered that prolonged dark incubation is critical for increasing shoot regeneration. Etiolated shoots regenerated in the dark were also found to facilitate subsequent visual selection of transformants by green fluorescent protein during Stevia transformation. Using the transformation method described herein, transgenic plants are prepared which overexpress the Stevia 1-deoxy-d-xylulose-5-phosphate synthase 1 (SrDXS1) and kaurenoic acid hydroxylase (SrKAH), both of which are required for SGs biosynthesis. Compared to control plants, the total SGs content in SrDXS1- and SrKAH-overexpressing lines were enhanced by up to 42%-54% and 67%-88%, respectively, showing a positive correlation with the expression levels of SrDXS1 and SrKAH. Furthermore, their overexpression did not stunt the growth and development of the transgenic Stevia plants. The invention described herein represents the first successful case of genetic manipulation of SGs biosynthetic pathway in Stevia and also demonstrates the potential of metabolic engineering towards producing Stevia with improved SGs yield.

Steviol glycosides (SGs) are extracted from the leaves of Stevia rebaudiana for use as a natural sweetener. Among these SGs, stevioside is most abundant in leaf extracts followed by rebaudioside A (Reb A). However, Reb A is of particular interest because of its sweeter and more pleasant taste compared to stevioside. Therefore, the development of new Stevia varieties with a higher Reb A to stevioside ratio would be desirable for the production of higher quality natural sweeteners. As described herein, transgenic Stevia plants overexpressing Stevia UDP-glycosyltransferase 76G1 (SrUGT76G1) that is known to convert stevioside to Reb A through 1,3-β-D-glucosylation were obtained. Interestingly, by overexpressing SrUGT76G1, the Reb A to stevioside ratio was drastically increased from 0.30 in wild type (WT) plants up to 1.55 in transgenic lines without any significant changes in total SGs content. This was contributed by a concurrent increase in Reb A content and a decrease in stevioside content. Additionally, an increase in the Reb C to dulcoside A ratio was seen in the SrUGT76G1-overexpression lines. Using the glutathione S-transferase-tagged SrUGT76G1 recombinant protein for an in vitro glycosyltransferase assay as shown herein, it was further demonstrated that Reb C can be produced from the glucosylation of dulcoside A by SrUGT76G1. Transgenic Stevia plants having higher Reb A to stevioside ratio were visually indistinguishable from WT plants. Taken together, the overexpression of SrUGT76G1 in Stevia is an effective way to generate new Stevia varieties with higher proportion of the more preferred Reb A without compromising on plant development.

Unless defined otherwise, all technical and scientific terms used herein have the same meaning as is commonly understood by one of skill in the art to which the invention belongs.

The term “about” or “approximately” means within a statistically meaningful range of a value. Such a range can be within an order of magnitude, preferably within 50%, more preferably within 20%, more preferably still within 10%, and even more preferably within 5% of a given value or range. The allowable variation encompassed by the term “about” or “approximately” depends on the particular system under study, and can be readily appreciated by one of ordinary skill in the art.

“Constitutive promoter” refers to a promoter which is capable of causing a gene to be expressed in most cell types at most. A “strong constitutive promoter” refers to a constitutive promoter that drives the expression of a mRNA to the top 10% of any mRNA species in any given cell.

“1-deoxy-D-xylulose 5-phosphate synthase 1” refers to the activity associated with a polypeptide, either a full length or a fragment, that is capable of catalyzing or partially catalyzing the condensation of pyruvate and d-glyceraldehyde-3-phosphate into 1-deoxy-d-xylulose-5-phosphate (DXP). Preferably, the polypeptide is 1-deoxy-D-xylulose 5-phosphate synthase 1 (DXS1), or a fragment thereof that is capable of catalyzing or partially catalyzing condensation of pyruvate and d-glyceraldehyde-3-phosphate into 1-deoxy-d-xylulose-5-phosphate.

“Ent-kaurenoic acid 13-hydroxylase activity” refers to the activity associated with a polypeptide, either a full length or a fragment, that is capable of catalyzing or partially catalyzing the conversion of ent-kaurenoic acid to steviol by mono-oxygenation. Preferably, the polypeptide is ent-kaurenoic acid 13-hydroxylase (KAH), or a fragment thereof that is capable of catalyzing or partially catalyzing the conversion of ent-kaurenoic acid to steviol by mono-oxygenation.

“UDP-glycosyltransferase 76G1 activity” refers to the activity associated with a polypeptide, either a full length or a fragment, that is capable of catalyzing or partially catalyzing the conversion of stevioside to rebaudioside A through 1,3-β-D-glucosylation. Preferably, the polypeptide is UDP-glycosyltransferase 76G1 (UGT76G1), or a fragment thereof that is capable of catalyzing or partially catalyzing the conversion of stevioside to rebaudioside A through 1,3-β-D-glucosylation.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence.

As used herein, “gene” refers to a nucleic acid sequence that encompasses a 5′ promoter region associated with the expression of the gene product, any intron and exon regions and 3′ or 5′ untranslated regions associated with the expression of the gene product.

As used herein, “genotype” refers to the genetic constitution of a cell or organism.

The term “heterologous” or “exogenous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous or exogenous protein indicates that the protein comprises two or more subsequences that are not found in the same relationship to each other in nature (e.g., a fusion protein).

“Inducible promoter” refers to a promoter which is capable of directly or indirectly activating transcription of one or more DNA sequences or genes in response to an inducer. The inducer can be a chemical agent such as a protein, metabolite, growth regulator, herbicide or phenolic compound or a physiological stress, such as that imposed directly by heat, cold, salt or toxic elements or indirectly through the action of a pathogen or disease agent such as a virus or other biological or physical agent or environmental condition.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Operable linkage” or “operably linked” or “operatively linked” as used herein is understood as meaning, for example, the sequential arrangement of a promoter and the nucleic acid to be expressed and, if appropriate, further regulatory elements such as, for example, a terminator, in such a way that each of the regulatory elements can fulfill its function in the recombinant expression of the nucleic acid to make dsRNA. This does not necessarily require direct linkage in the chemical sense. Genetic control sequences such as, for example, enhancer sequences, can also exert their function on the target sequence from positions which are somewhat distant, or indeed from other DNA molecules (cis or trans localization). Preferred arrangements are those in which the nucleic acid sequence to be expressed recombinantly is positioned downstream of the sequence which acts as promoter, so that the two sequences are covalently bonded with one another. Regulatory or control sequences may be positioned on the 5′ side of the nucleotide sequence or on the 3′ side of the nucleotide sequence as is well known in the art.

“Over-expression” or “overexpression” refers to the production of a gene product in transgenic organisms that exceeds levels of production in normal, control or non-transformed organisms. “Overexpression construct” refers to at nucleic acid construct useful for the overexpression of a gene product in a transgenic organism.

As used herein, “phenotype” refers to the detectable characteristics of a cell or organism, which characteristics are the manifestation of gene expression.

The terms “polynucleotide,” “nucleic acid” and “nucleic acid molecule” are used interchangeably herein to refer to a polymer of nucleotides which may be a natural or synthetic linear and sequential array of nucleotides and/or nucleosides, including deoxyribonucleic acid, ribonucleic acid, and derivatives thereof. It includes chromosomal DNA, self-replicating plasmids, infectious polymers of DNA or RNA and DNA or RNA that performs a primarily structural role. Unless otherwise indicated, nucleic acids or polynucleotide are written left to right in 5′ to 3′ orientation, Nucleotides are referred to by their commonly accepted single-letter codes. Numeric ranges are inclusive of the numbers defining the range.

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers Amino acids may be referred to by their commonly known three-letter or one-letter symbols. Amino acid sequences are written left to right in amino to carboxy orientation, respectively. Numeric ranges are inclusive of the numbers defining the range.

“Progeny” comprises any subsequent generation of a plant.

“Promoter” refers to a nucleic acid fragment capable of controlling transcription of another nucleic acid fragment.

“Promoter functional in a plant” or “promoter operable in a plant” is a promoter capable of controlling transcription in plant cells whether or not its origin is from a plant cell.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” or “nucleic acid construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein. In several embodiments described herein, a recombinant DNA construct may also be considered an “over expression DNA construct” or “overexpression nucleic acid construct”.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

The term “steviol” refers to the diterpenoic compound hydroxy-ent-kaur-16-en-β-ol-19-oic acid, which is the hydroxylated form of the compound termed “ent-kaurenoic acid”, which is ent-kaur-16-en-19-oic acid.

The term “steviol glycoside” refers to any of the glycosides of the aglycone steviol including, but not limited to stevioside, rebaudioside A, rebaudioside B, rebaudioside C, rebaudioside D, rebaudisode E, rebaudisode F, dulcoside, rubusoside, steviolmonoside, steviolbioside, and 19-O-β-glucopyranosol-steviol

“Transformation” as used herein refers to both stable transformation and transient transformation.

A “transformed cell” is any cell into which a nucleic acid fragment (e.g., a recombinant DNA construct) has been introduced.

“Transgenic plant” includes reference to a plant which comprises within its genome a polynucleotide not present in a wild type plant. The polynucleotide may be a heterologous polynucleotide or it may be an overexpression construct. For example, the polynucleotide is stably integrated within the genome such that the polynucleotide is passed on to successive generations. The polynucleotide may be integrated into the genome alone or as part of a recombinant DNA construct. “Transgenic plants” also include reference to plants which comprise more than one polynucleotide not present in a wild type plant within their genome. A “transgenic plant” encompasses all descendants which continue to harbor the polynucleotide.

Thus, in one aspect, the present invention provides an efficient and reproducible method for regeneration of Stevia. In some embodiments, explants are obtained from the second and third leaves of in vitro propagated Stevia plants. In some embodiments, the explants are cultured on callus induction medium (CIM) which comprises MS mineral salts, MS vitamins, sucrose and 6-benzylaminopurine (BA) and 3-indoleacetic acid (IAA) as plant hormones for a period of time for the formation of callus. At least 89% the explants have callus formation with compact callus condition when BA and IAA are used as the plant hormones. In some embodiments, the amount of BA in the CIM is about 1.0 mg/L. In some embodiments the amount of IAA in the CIM is about 0.5 mg/L. In some embodiments, the amount of sucrose is about 3%. In some embodiments, the culturing on the CIM is done in the dark. In some embodiments, the CIM is a solid medium. The CIM can be solidified using conventional plant tissue culturing solidifying agents such as agar or phytagel, preferably agar. In some embodiments, the explants are cultured on the CIM for three to four weeks for the production of callus.

In some embodiments, callus tissue is then transferred to a shoot induction medium (SIM) which comprises MS mineral salts, MS vitamins, sucrose and BA and IAA as plant hormones for a period of time for the formation of shoots. A higher BA to IAA ratio in the SIM is more efficient for promoting shoot regeneration. In some embodiments, the amount of BA in the SIM is about 1.0 mg/L to about 2.0 mg/L. In some embodiments, the amount of IAA in the SIM is about 0.25 mg/L to about 0.5 mg/L. In some embodiments, the amount of sucrose is about 3%. In some embodiments, the culturing on the SIM is done in the dark. In some embodiments, the SIM is a solid medium. The SIM can be solidified using conventional plant tissue culturing solidifying agents such as agar or phytagel, preferably agar. In some embodiments, the callus is subcultured on the SIM every three to four weeks for the production of shoots.

In some embodiments, the shoots are then transferred to a rooting medium (RM) which comprises MS mineral salts, MS vitamins, sucrose and IAA as a plant hormone. In some embodiments, the amount of IAA in the RM is about 0.5 mg/L. In some embodiments, the amount of sucrose is about 3%. In some embodiments, the shoots are cultured on RM in a Light/Dark (LD) (16 h L/8 h D) plant growth chamber maintained at about 25° C. In some embodiments, the RM is a solid medium. The RM can be solidified using conventional plant tissue culturing solidifying agents such as agar or phytagel, preferably agar. In some embodiments, after rooting, the plantlets are transferred to potting soil mixed with sand.

In some embodiments, the explants are first cultured in a co-culturing medium (CCM) which comprises MS mineral salts, MS vitamins, sucrose and 2,4-dichlorophenoxyacetic acid (2,4-D) as a plant hormone prior to culturing on the CIM. In some embodiments, the amount of 2,4-D in the CCM is about 0.25 mg/L. In some embodiments, the amount of sucrose is about 3%. In some embodiments, the CCM further comprises acetosyringone. In some embodiments, the amount of acetosyringone is about 100 μM. In some embodiments, the culturing on CCM is done in the dark. In some embodiments, the explants are cultured on the CCM for about 3 days. In some embodiments, the CCM is a solid medium. Prior culturing on the CCM leads to the production of regenerated shoots which are healthier.

In some embodiments, the Stevia plant is a Stevia rebaudiana plant. In some embodiments, the Stevia rebaudiana plant is a Stevia rebaudiana Bertoni plant.

In some embodiments, the Stevia plants and regenerated Stevia plants are propagated and maintained in vitro by cutting and transferring apicals (apical tissue) onto RM every three to four weeks. In some embodiments, the in vitro plants are kept in a Light/Dark (LD) (16 h L/8 h D) plant growth chamber maintained at about 25° C. In some embodiments, after rooting, in vitro plants were transferred to potting soil mixed with sand and covered with a transparent plastic dome for hardening.

In another aspect, the present invention provides an efficient and reproducible method for Agrobacterium-mediated transformation of Stevia. In some embodiments, the Agrobacterium-mediated transformation of Stevia plants utilizes the same basic scheme as described above for the regeneration of Stevia plants. In some embodiments for transformation, the explants are first co-cultured with Agrobacterium cells on CCM prior to transfer to the CIM with subsequent transfers to the SIM and RM as described above. In some embodiments, the CCM described above for regeneration further comprises acetosyringone when used for culturing the Stevia plant explants and the Agrobacterium cells. In some embodiments, the amount of acetosyringone is about 100 μM. In some embodiments, the Agrobacterium cells contain a vector for the transfer of a nucleic acid construct to be integrated into the plant genome to the Stevia genome and stable integration therein. Suitable Agrobacterium strains and vectors are well known in the art and are suitable for the transformation of Stevia plants. In some embodiments, the Agrobacterium strain is AGL2 (U.S. Patent Application Publication No. 2012/0246759). In some embodiments, the Agrobacterium strain is AGL3 (U.S. Patent Application Publication No. 2012/0246759).

In some embodiments, the CIM described above for regeneration further comprises a selective agent and an Agrobacterium eradicant. In some embodiments, the SIM described above for regeneration further comprises a selective agent and an Agrobacterium eradicant. In some embodiments, conventional selective agents and conventional Agrobacterium eradicants can be used for the Agrobacterium-mediated transformation of Stevia plants. In some embodiments, a suitable Agrobacterium eradicant is cefotaxime.

Suitable selective agents are described below. In some embodiments, a selective agent produced by transgenic plant tissue is also used. In some embodiments, such a selective agent is an enhanced green fluorescent protein (GFP) gene. In some embodiments, the GFP gene is used in combination with a selective agent as described below present in the media. In some embodiments, the enhanced GFP gene is fused to an endoplasmic reticulum (ER) targeting signal (EgfpER) (Haseloff et al., 1997; Karim et al., 2002). In some embodiments, the enhanced GFP gene is introduced into the plant tissue using the same Agrobacterium cells that contains a nucleic acid construct to be integrated into the plant genome. The use of an enhanced GFP gene and a selective agent in the media permit concurrent and earlier selection of transformed callus and regenerated transgenic shoots. In some embodiments, the calli is screened for GFP, and calli showing GFP spots are transferred to SIM.

In some embodiments, the culturing on CCM, CIM and SIM are done in the dark as described above. In some embodiments, the CCM, CIM, SIM and RM are solid media as described above. In some embodiments, the Stevia plant is a Stevia rebaudiana plant. In some embodiments, the Stevia rebaudiana plant is a Stevia rebaudiana Bertoni plant. In some embodiments, an average of 90% of the explants formed calli that show at least a single GFP spot and about 5% of them developed GFP positive shoots using the transformation method described herein.

In some embodiments, the transgenic Stevia plants are propagated and maintained in vitro by cutting and transferring apicals (apical tissue) onto RM every three to four weeks. In some embodiments, the in vitro transgenic plants are kept in a Light/Dark (LD) (16 h L/8 h D) plant growth chamber maintained at about 25° C. In some embodiments, after rooting, in vitro transgenic plants are transferred to potting soil mixed with sand and covered with a transparent plastic dome for hardening. In some embodiments, when enhanced GFP is used, the in vitro propagated plants are monitored for the GFP signals emitted. In some embodiment, transgenic Stevia plants showing GFP expression in whole tissues are transferred to soil for hardening and grown in a greenhouse for further maintenance.

The DNA that is inserted (the DNA of interest) into Stevia plants is not critical to the transformation process. Generally the DNA that is introduced into a plant is part of a construct. The DNA may be a gene of interest, e.g., a coding sequence for a protein, or it may be a sequence that is capable of regulating expression of a gene, such as an antisense sequence, a sense suppression sequence or a miRNA sequence. The construct typically includes regulatory regions operatively linked to the 5′ side of the DNA of interest and/or to the 3′ side of the DNA of interest. A cassette containing all of these elements is also referred to herein as an expression cassette. The expression cassettes may additionally contain 5′ leader sequences in the expression cassette construct. The regulatory regions (i.e., promoters, transcriptional regulatory regions, and translational termination regions) and/or the polynucleotide encoding a signal anchor may be native/analogous to the host cell or to each other. Alternatively, the regulatory regions and/or the polynucleotide encoding a signal anchor may be heterologous to the host cell or to each other. See, U.S. Pat. No. 7,205,453 and U.S. Patent Application Publication Nos. 2006/0218670 and 2006/0248616. The expression cassette may additionally contain selectable marker genes. See, U.S. Pat. No. 7,205,453 and U.S. Patent Application Publication Nos. 2006/0218670 and 2006/0248616.

Generally, the expression cassette will comprise a selectable marker gene for the selection of transformed cells. Selectable marker genes are utilized for the selection of transformed cells or tissues. Usually, the plant selectable marker gene will encode antibiotic resistance, with suitable genes including at least one set of genes coding for resistance to the antibiotics spectinomycin, streptomycin, kanamycin, geneticin or hygromycin. Genes coding for antibiotic resistance include, but are not limited to the spectinomycin phosphotransferase (spt) gene coding for spectinomycin resistance, the neomycin phosphotransferase (nptII) gene encoding kanamycin or geneticin resistance, the hygromycin phosphotransferase (hpt or aphiv) gene encoding resistance to hygromycin, acetolactate synthase (als) genes. Alternatively, the plant selectable marker gene will encode herbicide resistance such as resistance to the sulfonylurea-type herbicides, glufosinate, glyphosate, ammonium, bromoxynil, imidazolinones, and 2,4-dichlorophenoxyacetate (2,4-D), including genes coding for resistance to herbicides which act to inhibit the action of glutamine synthase such as phosphinothricin or basta (e.g., the bar gene). See generally, WO 02/36782, U.S. Pat. No. 7,205,453 and U.S. Patent Application Publication Nos. 2006/0248616 and 2007/0143880, and those references cited therein. This list of selectable marker genes is not meant to be limiting. Any selectable marker gene can be used.

In some embodiments, a selective agent produced in the transgenic plant may be used. In some embodiments, such a selective agent is an enhanced green fluorescent protein (Zhang et al., 1996).

A number of promoters can be used in the practice of the invention. The promoters can be selected based on the desired outcome. That is, the nucleic acids can be combined with constitutive, tissue-preferred, or other promoters for expression in the host cell of interest. Such constitutive promoters include, for example, the core promoter of the Rsyn7 (WO 99/48338 and U.S. Pat. No. 6,072,050); the core CaMV35S promoter (Odell et al., 1985); rice actin (McElroy et al., 1990); ubiquitin (Christensen and Quail, 1989 and Christensen et al., 1992); pEMU (Last et al., 1991); MAS (Velten et al., 1984); ALS promoter (U.S. Pat. No. 5,659,026), and the like. Other constitutive promoters include, for example, those disclosed in U.S. Pat. Nos. 5,608,149; 5,608,144; 5,604,121; 5,569,597; 5,466,785; 5,399,680; 5,268,463; and 5,608,142.

Other promoters include inducible promoters, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. Other promoters include those that are expressed locally at or near the site of pathogen infection. In further embodiments, the promoter may be a wound-inducible promoter. In other embodiments, chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. In addition, tissue-preferred promoters can be utilized to target enhanced expression of a polynucleotide of interest within a particular plant tissue. Each of these promoters are described in U.S. Pat. Nos. 6,506,962, 6,575,814, 6,972,349 and 7,301,069 and in U.S. Patent Application Publication Nos. 2007/0061917 and 2007/0143880.

Where appropriate, the DNA of interest may be optimized for increased expression in the transformed plant. That is, the coding sequences can be synthesized using plant-preferred codons for improved expression. Methods are available in the art for synthesizing plant-preferred genes. See, for example, U.S. Pat. Nos. 5,380,831, 5,436,391, and 7,205,453 and U.S. Patent Application Publication Nos. 2006/0218670 and 2006/0248616.

In an additional aspect, the present invention provides transgenic Stevia plants having an enhanced content of steviol glycosides. In some embodiments, the Stevia plant is a Stevia rebaudiana plant. In some embodiments, the Stevia rebaudiana plant is a Stevia rebaudiana Bertoni plant. In some embodiments, the steviol glycosides are enhanced in transgenic Stevia plants by at least about 42%. In some embodiments, the steviol glycosides are enhanced in transgenic Stevia plants by up to about 88%.

In one embodiment, transgenic Stevia plants having an enhanced steviol glycosides content overexpress SrDXS1. In some embodiments, these transgenic plants are obtained using the Agrobacterium-mediated transformation method described herein to stably integrate a nucleic acid construct comprising a polynucleotide which encodes SrDXS1 in the genome of Stevia plants. In some embodiments, the SrDXS1 has the amino acid sequence set forth in SEQ ID NO:2. In some embodiments, the polynucleotide encoding SrDXS1 has the nucleotide sequence set forth in SEQ ID NO:1. In some embodiments, the polynucleotide encoding SrDXS1 has the nucleotide sequence set forth in nucleotides 335-2479 of SEQ ID NO:1. In some embodiments, the nucleic acid construct comprises the polynucleotide operably linked to 5′ and 3′ regulatory sequences known in the art, including those described herein. In some embodiments, a cauliflower mosaic virus (CaMV) 35S promoter is operatively linked to the polynucleotide. In some embodiments, the steviol glycosides include stevioside, Reb A, Reb C and dulcoside A.

In some embodiments, the steviol glycosides are enhanced by at least about 42% in transgenic Stevia plants overexpressing SrDXS1. In some embodiments, the steviol glycosides are enhanced by up to about 54% in transgenic Stevia plants overexpressing SrDXS1. In some embodiments, the steviol glycosides are enhanced by from about 42% to about 54% in transgenic Stevia plants overexpressing SrDXS1.

In another embodiment, transgenic Stevia plants having an enhanced steviol glycosides content overexpress SrKAH. In some embodiments, these transgenic plants are obtained using the Agrobacterium-mediated transformation method described herein to stably integrate a nucleic acid construct comprising a polynucleotide which encodes SrKAH in the genome of Stevia plants. In some embodiments, the SrKAH has the amino acid sequence set forth in SEQ ID NO:4. In some embodiments, the polynucleotide encoding SrKAH has the nucleotide sequence set forth in SEQ ID NO:3. In some embodiments, the polynucleotide encoding SrKAH has the nucleotide sequence set forth in nucleotides 1-1431 of SEQ ID NO:3. In some embodiments, the nucleic acid construct comprises the polynucleotide operably linked to 5′ and 3′ regulatory sequences known in the art, including those described herein. In some embodiments, a cauliflower mosaic virus (CaMV) 35S promoter is operatively linked to the polynucleotide. In some embodiments, the steviol glycosides include stevioside, Reb A, Reb C and dulcoside A.

In some embodiments, the steviol glycosides are enhanced by at least about 67% in transgenic Stevia plants overexpressing SrKAH. In some embodiments, the steviol glycosides are enhanced by up to about 88% in transgenic Stevia plants overexpressing SrKAH. In some embodiments, the steviol glycosides are enhanced by from about 67% to about 88% in transgenic Stevia plants overexpressing SrKAH.

In a further embodiment, transgenic Stevia plants having an enhanced steviol glycosides content overexpress one or more SrUGT genes, such as SrUGT76G1, SrUGT74G1, SrUGT85C2, and others. In some embodiments, these transgenic plants are obtained using the Agrobacterium-mediated transformation method described herein to stably integrate a nucleic acid construct comprising a polynucleotide which encodes SrUGT76G1 in the genome of Stevia plants. In some embodiments, the SrUGT76G1 has the amino acid sequence set forth in SEQ ID NO:30. In some embodiments, the polynucleotide encoding SrUGT76G1 has the nucleotide sequence set forth in SEQ ID NO:29. In some embodiments, the polynucleotide encoding SrUGT76G1 has the nucleotide sequence set forth in nucleotides 28-1404 of SEQ ID NO:29. In some embodiments, the nucleic acid construct comprises the polynucleotide operably linked to 5′ and 3′ regulatory sequences known in the art, including those described herein. In some embodiments, a cauliflower mosaic virus (CaMV) 35S promoter is operatively linked to the polynucleotide. In some embodiments, the steviol glycosides include stevioside, Reb A and Reb B.

In some embodiments, these transgenic plants are obtained using the Agrobacterium-mediated transformation method described herein to stably integrate a nucleic acid construct comprising a polynucleotide which encodes SrUGT74G1 in the genome of Stevia plants. In some embodiments, the SrUGT74G1 has the amino acid sequence set forth in SEQ ID NO:32. In some embodiments, the polynucleotide encoding SrUGT74G1 has the nucleotide sequence set forth in SEQ ID NO:31. In some embodiments, the polynucleotide encoding SrUGT74G1 has the nucleotide sequence set forth in nucleotides 1-1383 of SEQ ID NO:31. In some embodiments, the nucleic acid construct comprises the polynucleotide operably linked to 5′ and 3′ regulatory sequences known in the art, including those described herein. In some embodiments, a cauliflower mosaic virus (CaMV) 35S promoter is operatively linked to the polynucleotide.

In some embodiments, these transgenic plants are obtained using the Agrobacterium-mediated transformation method described herein to stably integrate a nucleic acid construct comprising a polynucleotide which encodes SrUGT85C2 in the genome of Stevia plants. In some embodiments, the SrUGT85C2 has the amino acid sequence set forth in SEQ ID NO:34. In some embodiments, the polynucleotide encoding SrUGT85C2 has the nucleotide sequence set forth in SEQ ID NO:33. In some embodiments, the polynucleotide encoding SrUGT85C2 has the nucleotide sequence set forth in nucleotides 1-1446 of SEQ ID NO:33. In some embodiments, the nucleic acid construct comprises the polynucleotide operably linked to 5′ and 3′ regulatory sequences known in the art, including those described herein. In some embodiments, a cauliflower mosaic virus (CaMV) 35S promoter is operatively linked to the polynucleotide.

In an additional aspect, the present invention provides transgenic Stevia plants having an enhanced content of rebaudiosides. In some embodiments, the Stevia plant is a Stevia rebaudiana plant. In some embodiments, the Stevia rebaudiana plant is a Stevia rebaudiana Bertoni plant. In some embodiments, the rebaudioside is Reb A. In some embodiments, the rebaudioside is Reb C. In some embodiments, the rebaudiosides are Reb A and Reb C. In some embodiments, the enhanced content of Reb A is expressed as a ratio of Reb A to stevioside. In wild type plants, the ratio of Reb A to stevioside is 0.3. In transgenic Stevia plants overexpressing UGT76G1, the ratio of Reb A to stevioside ranges from about 0.62 to about 1.55. That is, the ratio of Reb A to stevioside is enhanced by about 207% to about 517%. In some embodiments, the enhanced content of Reb C is expressed as a ratio of Reb C to dulcoside A. In wild type plants, the ratio of Reb C to dulcoside A is 1.79. In transgenic Stevia plants overexpressing UGT76G1, the ratio of Reb C to dulcoside A ranges from about 2.41 to about 3.97. That is, the ratio of Reb C to dulcoside is enhanced by about 135% to about 222%.

In one embodiment, transgenic Stevia plants having an enhanced rebaudioside content overexpress SrUGT76G1. In some embodiments, these transgenic plants are obtained using the Agrobacterium-mediated transformation method described herein to stably integrate a nucleic acid construct comprising a polynucleotide which encodes SrUGT76G1 in the genome of Stevia plants. In some embodiments, the SrUGT76G1 has the amino acid sequence set forth in SEQ ID NO:30. In some embodiments, the polynucleotide encoding SrUGT76G1has the nucleotide sequence set forth in SEQ ID NO:29. In some embodiments, the polynucleotide encoding SrUGT76G1has the nucleotide sequence set forth in nucleotides 28-1404 of SEQ ID NO:29. In some embodiments, the nucleic acid construct comprises the polynucleotide operably linked to 5′ and 3′ regulatory sequences known in the art, including those described herein. In some embodiments, a cauliflower mosaic virus (CaMV) 35S promoter is operatively linked to the polynucleotide. In some embodiments, the rebaudioside includes Reb A and Reb C.

In some embodiments, the enhanced content of Reb A in transgenic Stevia plants overexpressing SrUGT76G1 is expressed as a ratio of Reb A to stevioside. In wild type plants, the ratio of Reb A to stevioside is 0.3. In transgenic Stevia plants overexpressing UGT76G1, the ratio of Reb A to stevioside ranges from about 0.62 to about 1.55. That is, the ratio of Reb A to stevioside is enhanced by about 207% to about 517%. In some embodiments, the enhanced content of Reb C in transgenic Stevia plants overexpressing SrUGT76G1 is expressed as a ratio of Reb C to dulcoside A. In wild type plants, the ratio of Reb C to dulcoside A is 1.79. In transgenic Stevia plants overexpressing UGT76G1, the ratio of Reb C to dulcoside A ranges from about 2.41 to about 3.97. That is, the ratio of Reb C to dulcoside is enhanced by about 135 to about 222%.

Since the whole transcriptome of Stevia has been sequenced (Chen et al., 2014; Kim et al., 2015), transformation of Stevia is indispensable not only in functional genomics for elucidating crucial genes such as those involved in SGs biosynthesis and stress response, but also for metabolic engineering to fulfill commercial interests in producing SGs more efficiently. As described herein, a method for shoot regeneration from Stevia leaf explants was developed and then adapted for the Stevia transformation. Among the different regeneration conditions analyzed herein, Condition F (CCM: 0.25 mg/L 2,4-D, CIM: 1 mg/L BA+0.5 mg/L IAA and SIM: 2 mg/L BA+0.25 mg/L IAA) with incubation in continuous dark was the most ideal as approximately 53% of the starting explants have healthy regenerated shoots (Table 3). Even though Khan et al. (2014) previously reported regeneration frequency of nearly 90%, attempts to replicate their condition which is equivalent to the Condition A only achieved a regeneration rate of 5% (Table 3).

As noted above, a prolonged dark incubation improved significantly the rate of shoot regeneration (Table 2). Similar findings have been reported in other plants such as rice and citrus (Duran-Vila et al., 1992; Marutani-Hert et al., 2012). It has been suggested that increased reactive oxygen species (ROS) levels during light exposure inhibit shoot regeneration (Ikeuchi et al., 2016; Nameth et al., 2013), which may be the cause for the low shoot regeneration rate observed in Stevia explants under light exposure.

For the selection of transgenic shoots, concurrent visual and antibiotic selection was the most suitable for Stevia . 50 mg/L of kanamycin was insufficient to completely inhibit the regeneration of non-transgenic shoots but higher amounts of kanamycin also reduced overall regeneration rate. The use of GFP for visual selection allowed the easy identification of transgenic shoots without compromising regeneration rate and thus maximized transformation rate. Such concurrent antibiotic and visual selection have also been employed for efficient transformation of the rubber tree and the sweet chestnut (Corredoira et al., 2012; Leclercq et al., 2010). Relying on this concurrent selection strategy together with Condition F, a transformation rate of about 5% (Table 4) was achieved.

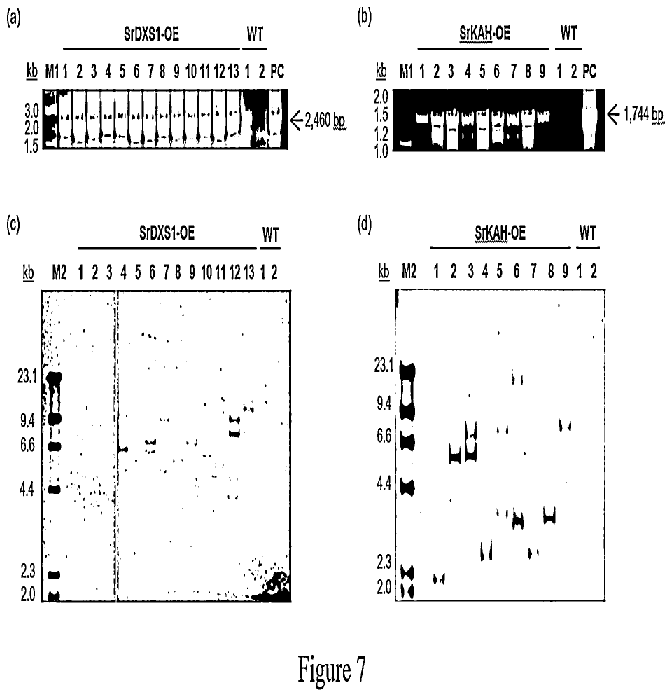

As shown herein, the stable integration of SrDXS1 or SrKAH into the genome of transgenic lines was confirmed by genomic PCR and Southern blot analyses. Notably, the genomic Southern blot analysis shows the first existence of transgene and its copy number in transgenic Stevia genome. Among transgenic Stevia plants, 46% of the SrDXS1-OE lines and 56% of the SrKAH-OE lines had a single copy of the transgene (FIGS. 7c and 7d).

For the study with next generation of transgenic Stevia lines, harvesting viable seeds under local environmental conditions was difficult. Even though lots of pollen grains attached to the stigma of the flowers, transgenic and WT seeds that were collected were always empty and non-viable. Nevertheless, by in vitro cutting propagation, clones of the transgenic lines that do not show a reduction in expression levels of the transgene or SG content over time are continually obtained.

Metabolic engineering to increase desirable metabolites in plants can be done through increasing flux towards the relevant pathways by overexpressing rate-limiting enzyme genes in the pathway (Ara et al., 2009). As shown herein, the total SG content was increased by up to 54% in transgenic lines overexpressing SrDXS1 when compared to vector-only control. In Arabidopsis, upregulation of DXS elevated chlorophylls and carotenoids concentrations together with GA and abscisic acid content (Estévez et al., 2001). However, the overexpression of SrDXS1 in Stevia transgenic plants did not affect levels of chlorophylls, carotenoids and monoterpenes tested. This finding is not unique to Stevia as the overexpression of Arabidopsis DXS in spike lavender also led to the higher amount of essential oil but no changes in the chlorophylls and carotenoids levels (Munoz-Bertomeu et al., 2006). The difference in response to elevated DXS levels seemed to imply that in plants producing specialized secondary metabolites, excess precursors from the MEP pathway would be diverted to their biosynthesis instead of the biosynthesis of primary metabolites such as the phytohormones and chlorophylls that could have adverse effects on the growth and development of the transgenic plants.

Other common targets for metabolic engineering include the cytochrome P450s as they tend to catalyze rate-limiting and irreversible steps in pathways with high specificity (Renault et al., 2014). By overexpressing SrKAH, transgenic lines were generated that were up to 88% more abundant in total SGs. The expression levels of SrKAH were also found to be positively correlated to the SGs contents in the SrKAH-OE lines. However, steviol but not SGs was previously detected in the leaves of Arabidopsis by heterologous expression of SrKAH (Guleria et al., 2015). This result was likely due to the lack of UGTs that could glycosylate steviol. In contrast, steviol remained undetectable in the leaves of the Stevia SrKAH-OE lines, possibly due to rapid glycosylation of newly synthesized steviol by downstream UGTs for sequestration into the vacuoles to avoid its potential toxicity (Ceunen and Geuns, 2013). Overexpression of SrKAH in Arabidopsis also led to dwarfism and pollen abnormality, which is characteristic of plants with reduced GA levels (Guleria et al., 2015). This result was attributed to the diversion of precursors for GA towards steviol biosynthesis. However, there were no obvious phenotypes of GA deficiency in the transgenic Stevia plants overexpressing SrKAH produced herein. This result suggests that GA biosynthesis was differentially regulated from SGs biosynthesis in Stevia leaves.

Comparing high expressers of SrKAH-OE and SrDXS1-OE lines produced herein, the increase in total SGs content in the former was higher than the later. This result is likely due to SrKAH being situated further down in the SGs biosynthesis pathway allowing its upregulation to have a more direct effect on SGs production. Another possible explanation is that the increased precursors supply from SrDXS1 upregulation might be diverted to the production of other metabolites along the many steps in the pathway. There may also be other rate-limiting steps in the pathway restricting the increase in SGs production. Nevertheless, the overexpression of SrDXS1 increased SGs levels without any obvious unintended effects. SGs content could further be enhanced by the co-expression of SrKAH and SrDXS1. The elevated SrKAH activity would help divert the greater amount of precursors resulting from SrDXS1 overexpression towards SGs biosynthesis more efficiently, having a push and pull effect (George et al., 2015; Tai and Stephanopoulos, 2013). It is recognized that among the two most abundant SGs present in Stevia leaves, Reb A has a sweeter and more pleasant taste profile than stevioside (Hellfritsch et al., 2012). Hence, it is also be desirable to target the UGTs to engineer Stevia with higher Reb A to stevioside ratio.

In summary, the present invention provides effective methods for Stevia regeneration and transformation which has been demonstrated by the production of SrDXS1-OE and SrKAH-OE lines. These methods are an important tool for creating lines with overexpression or knockdown of genes from the Stevia RNA-seq database. Furthermore, these methods facilitate metabolic engineering of Stevia with greatly enhanced total SGs content and more pleasant tasting SGs including the minor SGs, Reb D and Reb M.

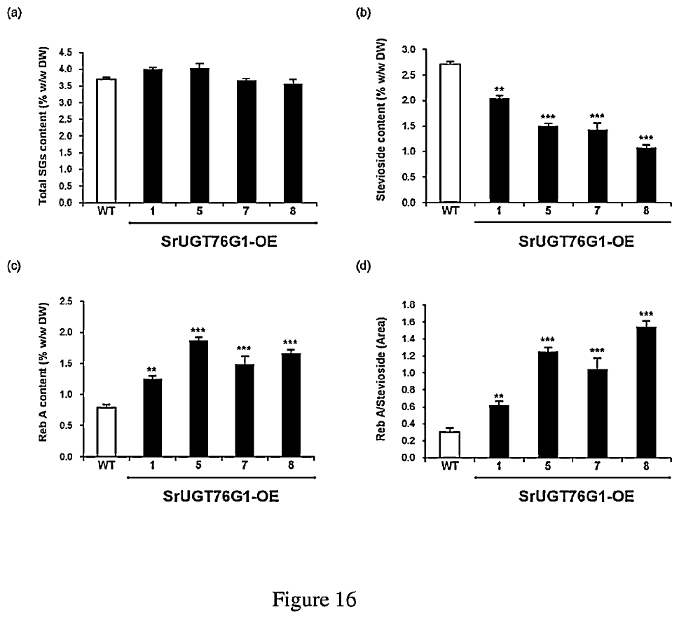

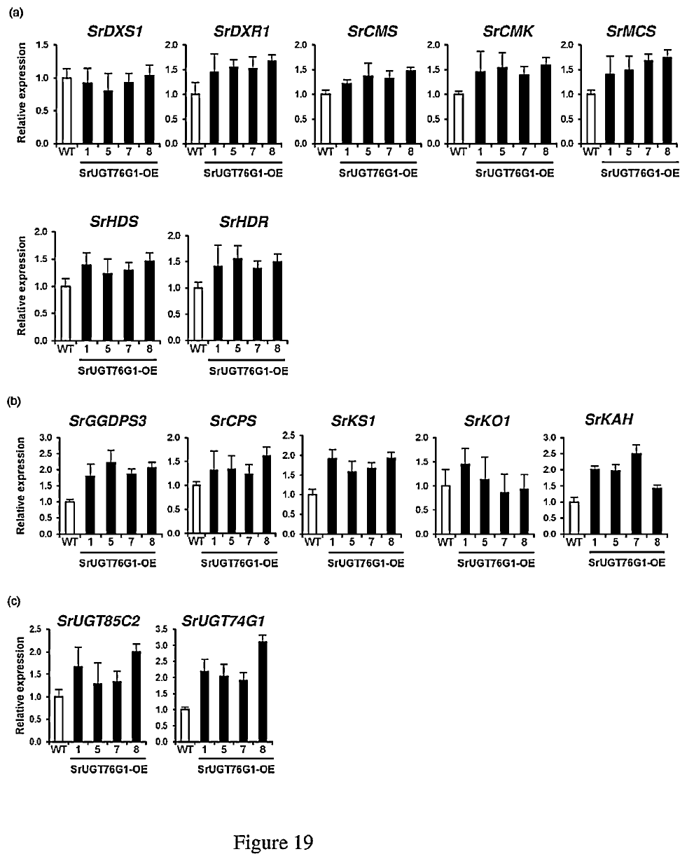

The pleasant taste of Reb A and its relative abundance in Stevia has made it one of the most commercially valuable SGs that can be extracted from Stevia leaves. However, as its abundance is less than stevioside, which has a relatively stronger bitter aftertaste, it is desirable to generate new cultivars with higher Reb A levels. As shown herein, the proportion of Reb A could be increased through the overexpression of SrUGT76G1, and its content in these transgenic lines reached concentrations of up to 1.87% (w/w dried weight (DW)) compared to the 0.79% (w/w DW) in the WT control. It is most likely a result of the increased conversion from stevioside which fell from 2.71% (w/w DW) in the WT down to 1.07% (w/w DW) in the transgenic lines (FIG. 16b). Moreover, the total content of the four most abundant SGs, stevioside, Reb A, Reb C and dulcoside A was not changed in the SrUGT76G1-OE lines (FIG. 16a). This suggests that SrUGT76G1 overexpression enhances the conversion of stevioside to Reb A present in Stevia but does not trigger a general increase in carbon flux towards SGs biosynthesis. This is supported by the lack of significant changes in transcript abundance of other genes in the SGs biosynthesis pathway following the overexpression of SrUGT76G1 (FIGS. 19a-19c).

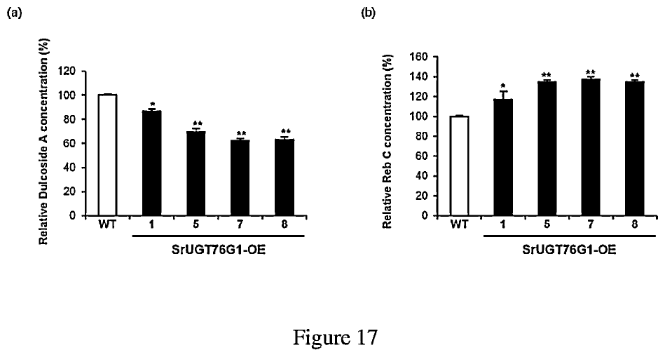

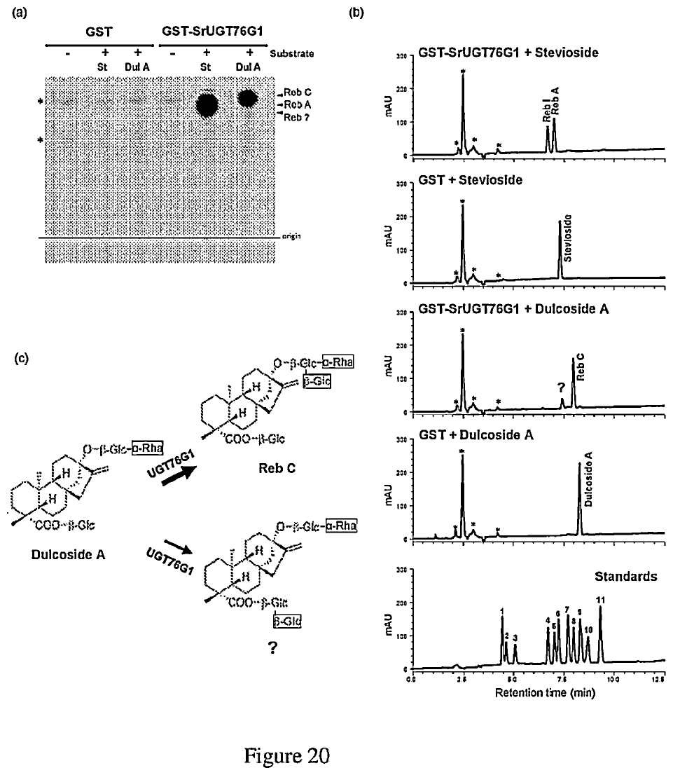

Other than a change in the Reb A/stevioside ratio, an increase in Reb C content relative to dulcoside A (FIGS. 17a and 17b) was also observed. It was previously suggested that Reb A and Reb C might be formed by the same or very closely linked enzyme because their proportions in the next generation are positively correlated (Brandle, 1999). Although it was discovered that SrUGT76G1 could convert stevioside to Reb A, the biosynthesis of Reb C remained unclear (Richman et al., 2005). However, Reb A and Reb C can be produced from the 1,3-glucosylation on the C₁₃-positioned glucose of stevioside and dulcoside A, respectively. By carrying out in vitro assays using purified recombinant SrUGT76G1 and dulcoside A, Reb C was detected as a product (FIGS. 20a and 20b). This confirms that Reb A and Reb C could certainly be synthesized by a common enzyme which is identified herein to be SrUGT76G1.

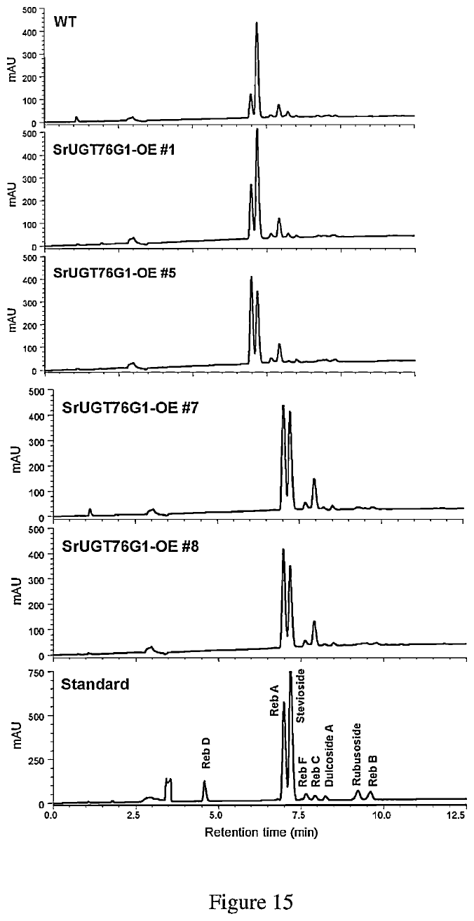

Plant UGTs have the potential to accept a broad range of substrates but show regiospecificity (Hansen et al., 2003). Earlier in vitro assays with SrUGT76G1 showed that it could carry out 1,3-glucosylation at the C₁₃- or C₁₉-positioned glucose of several substrates including, stevioside, steviobioside, rubusoside, Reb A, Reb D, Reb E, and Reb G (Olsson et al., 2016). The identification herein of dulcoside A as a substrate of SrUGT76G1 further adds to this list. However, these in vitro conversions did not all translate into in vivo observations in the Stevia with SrUGT76G1 overexpression. In particular, although Reb A was converted by SrUGT76G1 into Reb I in vitro (FIG. 20b; Olsson et al., 2016), Reb I was not detectable in the SrUGT76G1-OE lines despite their elevated Reb A levels (FIG. 15). Furthermore, as shown herein, a significant increase in Reb B content was not detected in the SrUGT76G1-OE lines even though steviobioside, which is a precursor to stevioside, could be converted by SrUGT76G1 into Reb B in vitro (Olsson et al., 2016). This suggests that on top of regiospecificity, other factors such as compartmentalization and enzyme affinity can influence the substrate specificity of SrUGT76G1 in vivo. However, such differences between in vitro and in vivo function of UGTs are not limited to SrUGT76G1 in Stevia. For example, AtUGT73C6 was found to only have flavonol-3-O-glycoside-7-O-glucosyltransferase activity in Arabidopsis but it could also glucosylate isoflavones and flavanoid aglycones in vitro (Jones et al., 2003; Bowles et al., 2005). However, the possibility that the concentrations of several substrates for SrUGT76G1 such as rubusoside, Reb E and Reb G are present only in minute amounts in Stevia leaves should not be excluded.

With changes in the proportion of the major SGs in the SrUGT76G1-OE lines, the total extract from the leaves is expected to have improved taste. By considering Reb A and stevioside that together make up more than 90% of all SGs in the leaves, Reb A, which is perceived to be sweeter than stevioside, is increased by up to 137% in contrast to the decrease of up to 61% in stevioside content in the SrUGT76G1-OE lines. Even among the two other major SGs that are considered less desirable due to their strong bitter taste, Reb C, which is slightly sweeter and less bitter, had an increase of up to 38% in content compared to the similar extent of decrease in dulcoside A. Therefore, the overexpression of SrUGT76G1 in Stevia would efficiently enhance the taste of Stevia leaf extracts.

In summary and as shown herein, other than converting stevioside to Reb A, SrUGT76G1 can also carry out 1,3-glucosylation on dulcoside A to produce Reb C both in vitro and in the Stevia plant. Since both these conversions lead to an increase in the proportion of the more pleasant tasting SG within each pair, SrUGT76G1 overexpression in the Stevia plant serves as an effective way to generate new varieties with chemotypes that are more commercially valuable.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, New York); Sambrook et al., 1989, Molecular Cloning, 2nd Ed. (Cold Spring Harbor Laboratory Press, Cold Spring Harbor, New York); Sambrook and Russell, 2001, Molecular Cloning, 3rd Ed. (Cold Spring Harbor Laboratory Press, Cold Spring Harbor, New York); Ausubel et al., 1992), Current Protocols in Molecular Biology (John Wiley & Sons, including periodic updates); Glover, 1985, DNA Cloning (IRL Press, Oxford); Russell, 1984, Molecular biology of plants: a laboratory course manual (Cold Spring Harbor Laboratory Press, Cold Spring Harbor, N.Y.); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, 1988, Antibodies, (Cold Spring Harbor Laboratory Press, Cold Spring Harbor, New York); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6^{th} Edition, Blackwell Scientific Publications, Oxford, 1988; Fire et al., RNA Interference Technology: From Basic Science to Drug Development, Cambridge University Press, Cambridge, 2005; Schepers, RNA Interference in Practice, Wiley—VCH, 2005; Engelke, RNA Interference (RNAi): The Nuts & Bolts of siRNA Technology, DNA Press, 2003; Gott, RNA Interference, Editing, and Modification: Methods and Protocols (Methods in Molecular Biology), Human Press, Totowa, N.J., 2004; Sohail, Gene Silencing by RNA Interference: Technology and Application, CRC, 2004.

### EXAMPLES

The present invention is described by reference to the following Examples, which are offered by way of illustration and are not intended to limit the invention in any manner. Standard techniques well known in the art or the techniques specifically described below were utilized.

### Example 1

### Materials and Methods for Examples 2-7

Plant materials and growth condition: Stevia rebaudiana Bertoni was propagated and maintained in vitro by cutting and transferring apicals onto fresh rooting medium (RM) containing Murashige & Skoog (MS) medium with 6.5 g/L agar and 0.5 mg/L of IAA every 3-4 weeks. The in vitro plants were kept in a Light/Dark (LD) (16 h L/8 h D) plant growth chamber maintained at 25° C. After rooting, they were transferred to potting soil mixed with sand and covered for one week with a transparent plastic dome for hardening.

Stevia tissue culture: The second and third leaves (cut into ˜5×5 mm pieces) from sterile 2-3 week-old in vitro propagated plants were used as the explant source for Stevia tissue culture and transformation. 40 pieces of explants were incubated on MS media with six different combinations (Conditions A-F, Table 1) of plant growth regulators under continuous darkness unless otherwise specified. Explants placed on callus induction medium (CIM) for three weeks were assessed for calli formation rates and transferred onto shoot induction medium (SIM) for another three weeks to evaluate the percentage of explants with regenerated shoots. One-way analysis of variance (ANOVA) was used to evaluate for differences in the callus formation and regeneration rates between the Conditions (Sahoo et al., 2011).

TABLE 1
Cytokinin and Auxin Combinations Tested for Callus
Induction and Shoot Regeneration from Stevia Leaf Explants

| Condition | CCM¹ (mg/L) | CIM (mg/L) | SIM (mg/L) |
|---|---|---|---|
| A | — | BA 1 + NAA 2 | BA 1 + NAA 2 |
| B | — | BA 1 + NAA 0.5 | BA 1 + NAA 0.5 |
| C | — | BA 1 + IAA 2 | BA 1 + IAA 2 |
| D | — | BA 1 + IAA 0.5 | BA 1 + IAA 0.5 |
| E | 2,4-D 0.25 | BA 1 + IAA 0.5 | BA 1 + IAA 0.5 |
| F | 2,4-D 0.25 | BA 1 + IAA 0.5 | BA 2 + IAA 0.25 |
| F-light | 2,4-D 0.25 | BA 1 + IAA 0.5² | BA 2 + IAA 0.25² |

¹Co-cultivation medium (CCM).
²Explants were incubated under light with 16 h L/8 h D photoperiod. BA, 6-benzylaminopurine; NAA, 1-naphthaleneacetic acid; IAA, 3-indoleacetic acid; 2,4-D, 2,4-dichlorophenoxyacetic acid.

Functional complementation assay for SrDXSs in Escherichia coli mutant: SrDXS1, SrDXS2, SrDXS3, and SrDXS4 amplified from Stevia cDNA using primers listed in Table 2 were cloned into the pDONR221 and followed by recombination into the pDEST17 using Gateway cloning technology (Invitrogen). The resulting pDEST17-SrDXS constructs were transformed into an E. coli dxs⁻ strain defective in DXS activity. For complementation assay, the transformed cells were streaked out on Luria-Bertani (LB) agar plates with 1 mM of mevalonate (MVA) or without MVA and incubated overnight at 37° C. AtDXS1 and pDEST17 transformed into the E. coli dxs⁻ strain were used as positive and negative controls, respectively.

TABLE 2
Primers

| | | |
|---|---|---|
| | Forward sequence (F) | Reverse sequence (R) |
| Name | (SEQ ID NO:) | (SEQ ID NO:) |

For Gateway cloning

| | | |
|---|---|---|
| SrDXS1 | AAAAAGCAGGCTTCATGGCGATTTGTGC | AGAAAGCTGGGTGTGACATAACCTCCAGAGC |
| | CTTTGCATTCCCG (5) | CTCTCGG (6) |
| SrDXS2 | AAAAAGCAGGCTTCATGGCTTTATGTGG | AGAAAGCTGGGTGTAATACATTGACAGCATG |
| | TGCTTTGAAGGGTG (7) | TAGCATCTCCTTGC (8) |
| SrDXS3 | AAAAAGCAGGCTTCATGACTACTGCTTC | AGAAAGCTGGGTGACACATCAAAAGAAGAGC |
| | TGCACATTGTTCTTTGG (9) | TTCACGGGTTC (10) |
| SrDXS4 | AAAAAGCAGGCTTCATGGCGGTTGCAGG | AGAAAGCTGGGTGCATTATTGATTTGTAT |
| | ATCGACCATGAA (11) | TGAAGTGCTTCTTTAGGT (12) |
| SrKAH | AAAAAGCAGGCTTCATGATTCAAGTTCT | AGAAAGCTGGGTGTCAAACTTGATGGGGATG |
| | AACACCGATCC (13) | AAGACG (14) |
| SrKAH_P2 | AAAAAGCAGGCTTCGGAGCAGCTATCAG | AGAAAGCTGGGTGTCAAACTTGATGGGGATG |
| | GATTGAACTA (15) | AAGACG (16) |

For RT-PCR

| | | |
|---|---|---|
| SrDXS1 | GCAACACTGTCGGAGAGAGGTG (17) | CTGTTAACTCCACCACACCAAGAC (18) |
| SrKAH | GAGCAACTAGAGATATCGAAGACG (19) | CACTCCAGTGTAGCTTCCATCCT (20) |
| SrActin | TCTTGATCTTGCTGGTCGTG (21) | GAGCAAGAACTTGAAACCGC (22) |

For confirmatory PCR in transgenic lines

| | | |
|---|---|---|
| SrDXS1-OE | TAGAGAGGCCTACGCGGCAGGT (23) | AGAAAGCTGGGTGTGACATAACCTCCAGAG |
| | | CCTCTCGG (24) |
| SrKAH-OE | TAGAGAGGCCTACGCGGCAGGT (25) | AGAAAGCTGGGTGTCAAACTTGATGGGGAT |
| | | GAAGACG (26) |

For Southern blot probes

| | | |
|---|---|---|
| NptII | ATGATTGAACAAGATGGATTGCACGCA | TCAGAAGAACTCGTCAAGAAGGCGATAG |
| | G(27) | (28) |

Subcellular localization of SrDXS1: The C-terminal YFP-tagged SrDXS1 construct was transformed into the Agrobacterium strain GV3101. The Agrobacterium suspension was infiltrated into the leaves of 4-week-old N. benthamiana plants and incubated at 24° C. under LD photoperiod for three days before excision and mounting on slides for observation under a CLSM (Carl Zeiss LSM 5 Exciter, Germany). Argon laser at 514 nm was used to excite YFP, and the bandpass and long pass were set at 500 to 550 nm and 560 nm, respectively. Image processing was done on LSM Image Browser.

Vector construction for Stevia transformation: The full-length ORF_{S} of SrDXS1 (accession number: KT276229; Kim et al., 2015) and SrKAH (accession number, ACD93722; Guleria et al., 2015; Wang et al., 2016) were PCR-amplified from cDNA derived from Stevia leaves using primers listed in Table 2. PCR products were cloned into pK7WG2D using Gateway technology (Invitrogen) to generate pK7WG2D-SrDXS1 and pK7WG2D-SrKAH. All clones were confirmed by sequencing.

Stevia transformation: Vector constructs were transformed into the Agrobacterium strain AGL2. For co-cultivation, Agrobacterium at log phase was pelleted and resuspended in MS supplemented with 100 μM of acetosyringone to OD₆₀₀ of 0.4-0.6. The explants were incubated with the Agrobacterium suspension for 30 min with occasional gentle shaking and then placed on CCM (0.25 mg/L 2,4-D) supplemented with 100 μM acetosyringone at 22° C. for 3 days in the dark. Following co-cultivation, the explants were washed twice with sterile deionized H₂O and once in MS media supplemented with 300 mg/L cefotaxime by vigorous shaking before soaking in MS media with cefotaxime for another 20 min. The washed explants were placed on CIM (1 mg/L BA+0.5 mg/L IAA) supplemented with 125 mg/L cefotaxime and 50 mg/L kanamycin for the next 3-4 weeks at 25° C. in the dark for callus induction. The calli were screened under a fluorescence stereomicroscope (Leica, Germany) and those showing GFP spots were transferred to SIM (2 mg/L BA+0.25 mg/L IAA) supplemented with 125 mg/L cefotaxime and 50 mg/L kanamycin and subcultured every 3-4 weeks. Regenerated shoots from calli emitting GFP signals were transferred onto RM supplemented with 125 mg/L of cefotaxime. Transformation efficiency of this protocol was tested using Agrobacterium harboring pK7WG2D in triplicates on 200 pieces of explants.

Verification of transgenic Stevia plants by genomic PCR and Southern blot analysis: Genomic DNA (gDNA) was extracted from approximately 600 mg of Stevia leaves using cetyltrimethylammonium bromide (CTAB)-based extraction method (Rogers and Bendich, 1989). The final gDNA pellet was washed with ice-cold 75% ethanol and dissolved in water.

PCR amplification was carried out from 100 ng of gDNA extracted from each line of transgenic Stevia to check for the presence of T-DNA using forward primers specific to the CaMV 35S promoter and reverse primers specific to the 3′-end of SrDXS1 or SrKAH (Table 2).

Southern blot analysis for detection of transgene integrations and copy number was performed using a digoxygenin (DIG)-labelled probe specific to the full-length nptII (Roche). The purity of the synthesized probes was checked by electrophoresis on a 1% agarose gel. gDNAs extracted from the SrDXS1-OE and SrKAH-OE lines were digested with HindIII and XBaI, respectively. After digestion, the fragments were resolved on a 0.8% agarose gel together with DIG-labelled DNA molecular weight marker II (Roche). The agarose gel was treated with 0.2M HCl followed by denaturation solution (0.5M NaOH, 1.5M NaCl) and neutralization solution (1M Tris-Cl pH7.4, 1.5M NaCl) and transferred to a positively charged nylon membrane (Hybond-N+, GE healthcare life sciences) in 20× SSC (3.0M NaCl, 0.3M sodium citrate, pH 7.0). After the transfer, UV-crosslinking was carried out using Stratalinker 2400 (Stratagene, USA). Then, DIG-based Southern blot hybridization was performed according to manufacturer's instructions (Roche). Chemiluminescence from the membrane was acquired with the ChemiDoc Touch Imaging System (Bio-Rad, USA).

Expression analysis by quantitative real-time PCR (qRT-PCR): Total RNA was extracted from homogenized Stevia leaves using the TRIzol reagent (Invitrogen) and then treated with deoxyribonuclease I (DNase I; Roche, USA) to avoid possible genomic DNA contamination. Total RNA concentration was measured using a Nanodrop spectrophotometer, ND-1000 (Thermo Fisher Scientific, USA). One μg of total RNA was used for cDNA synthesis with M-MLV Superscript II (Promega, USA).

qRT-PCR was performed using SYBR Premix Ex Taq II (Takara, Japan) on the synthesized cDNA. The gene-specific primers are listed in Table 2. The expression levels were quantified on Applied Biosystems (USA) 7900HT fast real-time PCR system. Stevia actin gene was used as an internal control for normalization. Specificity of the amplified PCR products was verified by regular PCR analysis and melting curve analysis on the qRT-PCR system. Biological and technical triplicates were carried out for each experiment.

Steviol glycosides content analysis by High Performance Liquid Chromatography: To analyze SGs content in the transgenic lines, leaves on the 6^{th} node were harvested from plants grown in the greenhouse for three weeks and dried overnight in a 60° C. oven. Sterile water was added at 1 mL per 10 mg of powdered sample and extraction was carried out twice by sonication in a 50° C. water bath for 20 min. The extracts were clarified by centrifugation at 3,000 g for 15 min and pooled. After filtering through a 0.45 μm filter, 1 mL of the sample was applied to a solid phase extraction (SPE) column C2 (Agilent, USA) and eluted in 1 mL of methanol:acetonitrile (50:50, v/v). Eluted samples were analyzed on Shidmadzu Nexera X2 ultra-high performance liquid chromatography (UHPLC) system as described previously (Kim et al., 2014).

Chlorophylls and total carotenoids analysis: To analyze the chlorophylls and total carotenoids content in the transgenic lines, 200 mg of leaves homogenized in liquid nitrogen was extracted twice with 2 ml of 100% methanol. Extraction was carried out at room temperature for 1 h in the dark with constant shaking Methanol fraction from both extracts was pooled and diluted 5 folds before their absorbance values at wavelengths 666 nm, 653 nm and 470 nm were determined using an Infinite M2000 microplate reader (Tecan, Switzerland). The relative amount of chlorophyll a, chlorophyll b and total carotenoids were calculated from their absorbance values using previously reported formula (Lichtenthaler and Wellburn, 1983).

Monoterpene content analysis by GC-MS: Leaves harvested from the 4^{th} and 5^{th} nodes of Stevia plants grown in the greenhouse for three weeks were homogenized in liquid nitrogen. Approximately 350 mg of leaf powder was extracted with 350 μL of ethyl acetate containing 20 μg/mL of camphor (Sigma-Aldrich) as an internal standard. After 3 h incubation at room temperature with constant shaking, the ethyl acetate fraction was transferred into a new tube and treated with anhydrous Na₂SO₄. The treated extracts were then filtered through a 0.45 gm nylon centrifuge tube (Corning, USA). The GC-MS analysis was performed on Agilent 7890A GC (Agilent Technologies, USA) system as described previously (Kim et al., 2015).

### Example 2

### Callus Induction and Shoot Regeneration from Stevia Leaf Explants

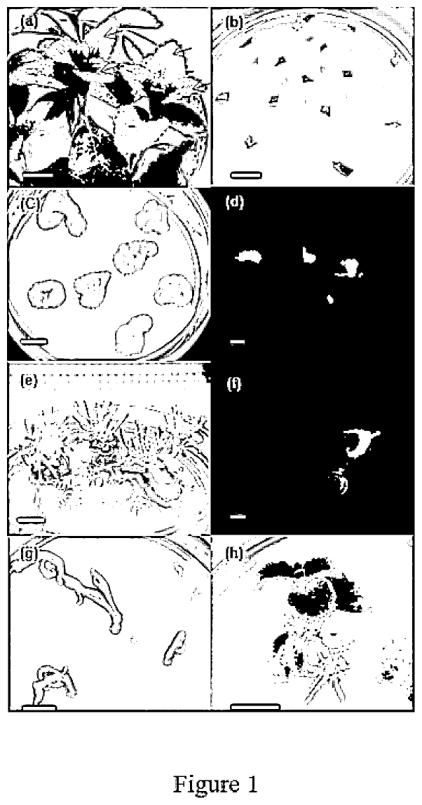

Plant transformation involves a few major steps namely, co-cultivation, callus induction, shoot regeneration and root regeneration, but all these steps require optimization to suit individual plants. To establish a standard transformation method for Stevia, the effects of different hormone combinations was investigated on callus induction and shoot regeneration by modifying existing procedures for tobacco transformation (Table 1; Horsch et al., 1985). The second and third leaves of in vitro cultured Stevia plants were chosen as the explant source (FIG. 1a).

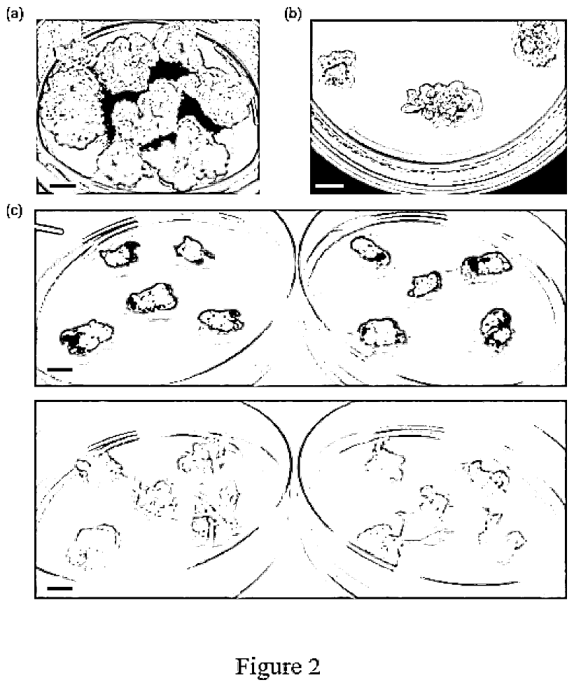

Plant growth regulators most frequently supplemented for shoot regeneration from Stevia leaf explants include 6-benzylaminopurine (BA) as the cytokinin and 1-naphthaleneacetic acid (NAA), or 3-indoleacetic acid (IAA) as the auxin (Aman et al., 2013; Anbazhagan et al., 2010; Patel and Shah, 2009). When explants were placed on BA with either NAA or IAA under long day photoperiod (LD, 16 h Light/8 h Dark), calli were induced on both media but with different appearance (FIGS. 2a and 2b). Shoot regeneration could also be observed from the calli on the BA+IAA media after six weeks but its frequency would be insufficient for successful transformation (FIG. 2b). It has been shown that a prolonged dark incubation promotes somatic embryogenesis from callus cultures of Stevia (Bespalhok-Filho and Hattori, 1997). Interestingly, drastic improvements in shoot regeneration from calli induced in the dark were found (FIG. 2c). Therefore, we subsequently incubated the explants under darkness during callus induction and shoot regeneration.

To compare the efficiency of BA with IAA or NAA on callus induction and shoot regeneration, four combinations (Conditions A-D in Table 1) with different concentration of NAA or IAA were designed. The difference in callus induction rates on four different callus induction media (CIM; Conditions A-D in Table 1) were not observed to be statistically significant (P-value: 0.099; Table 2). However, calli on CIM containing NAA (Conditions A and B) appeared friable while those on media containing IAA appeared compact (Conditions C and D; Table 2). Subsequently, calli maintained on NAA (Conditions A and B) had lower shoot regeneration rates than those on IAA (Conditions C and D; Table 3). Furthermore, it was found that a higher BA to IAA ratio (Condition D) was more efficient for promoting shoot regeneration (Table 3).

TABLE 3
Callus Induction and Regeneration Rates under the
Different Cytokinin and Auxin Combinations Listed in Table 1

| | Explants with | | Explants with | |
|---|---|---|---|---|
| | callus | Callus | regeneration | Shoot |
| Condition | formation (%) | condition | (%) | Condition |
| A | 87.4 ± 2.5 | Friable | 5.0 ± 1.4 | + |
| B | 99.2 ± 0.8 | Friable | 22.8 ± 2.6 | ++ |
| C | 89.1 ± 5.1 | Compact | 29.4 ± 2.9 | +++++ |
| D | 98.3 ± 0.8 | Compact | 65.8 ± 3.6 | ++++ |
| E | 95.0 ± 3.8 | Compact | 53.3 ± 5.1 | +++++ |
| F | 96.7 ± 3.3 | Compact | 53.3 ± 5.8 | +++++ |
| F-light | 95.8 ± 1.7 | Compact | 29.5 ± 7.7 | ++++ |

Values are mean ± SE of technical triplicates with n = 40.

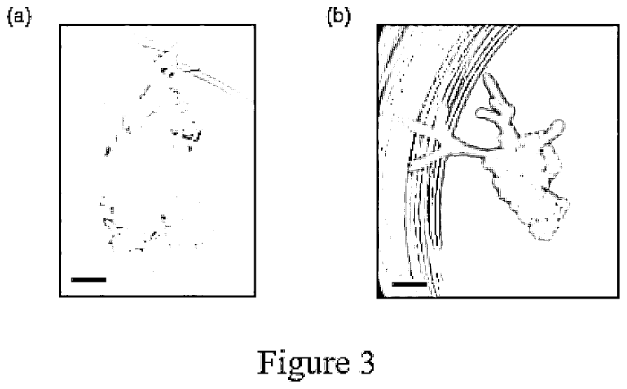

2,4-D is commonly used for the dedifferentiation of somatic cells (Gorst, 1999). Therefore, to further enhance regeneration rates under Condition D, Condition E was designed with an additional 3 d incubation on 0.25 mg/L 2,4-D (Table 1), which can also be used as the co-cultivation media (CCM) for Agrobacterium-mediated transformation. Although regeneration rates for Conditions E were similar to Condition D, the regenerated shoots were healthier (Table 3 and FIGS. 3a and 3b).

In general, a higher cytokinin to auxin ratio promotes shoot formation (Su et al., 2011). Condition E was further modified by doubling the cytokinin concentration to 2 mg/L and reducing the auxin concentration from 0.5 mg/L to 0.25 mg/L to form Condition F (Table 1). Under Condition F, rates for callus formation and shoot regeneration, and the shoot condition were comparable to those under Condition E (Table 3), but the number of regenerated shoots per callus clump was considerably higher (FIG. 1e). Next, Condition F was tested simultaneously under LD condition after the explants were transferred onto CIM (Condition F-light; Table 1) to verify the enhancement of shoot regeneration in the dark. Certainly, the percentage of explants with regenerated shoots was 1.8 times higher under Condition F (Table 3), confirming that dark incubation promotes shoot regeneration greatly. Therefore, Condition F was subsequently used for Stevia transformation.

### Example 3

### Stevia Transformation

To investigate the transformation efficiency using Condition F, Stevia leaf explants were co-cultivated on the CCM media containing acetosyringone with Agrobacterium harboring the pK7WG2D vector (Karimi et al., 2002), which contains a neomycin phosphotransferase (nptII) gene and an enhanced GFP gene fused to an endoplasmic reticulum targeting signal (EgfpER) to allow concurrent selection (FIG. 1b). FIGS. 1a-1h outline the overall procedures for Agrobacterium-mediated transformation of Stevia. The appearance of the calli and regenerated shoots on media are shown in FIGS. 1c and 1e, respectively. GFP signals from transgenic calli or regenerated shoots were monitored and selected under a fluorescence stereomicroscope (FIGS. 1d and 1f). For rooting, transgenic shoots were transferred onto rooting media (RM) and exposed to light for approximately one month (FIGS. 1g and 1h). Overall, it was found that on average, 90% of the explants formed calli that show at least a single GFP spot and nearly 5% of them developed GFP positive shoots after one month on SIM (Table 4).

TABLE 4
Transformation Rates of Stevia Leaf Explants under Condition F

| Transformed calli (%) | Transformed shoots (%) |
|---|---|
| 90.7 ± 2.8 | 4.6 ± 1.1 |

### Example 4

### Transformation of Stevia with SrDXS1 and SrKAH

DXS has been reported to play a rate-limiting role in the MEP pathway (Cordoba et al., 2009; Estévez et al., 2001; Lois et al., 2000), while Stevia KAH acts on kaurenoic acid as the committed step to SGs biosynthesis (Brandle and Telmer, 2007). Thus, it was hypothesized that their overexpression would lead to an increase the flux towards SGs production.

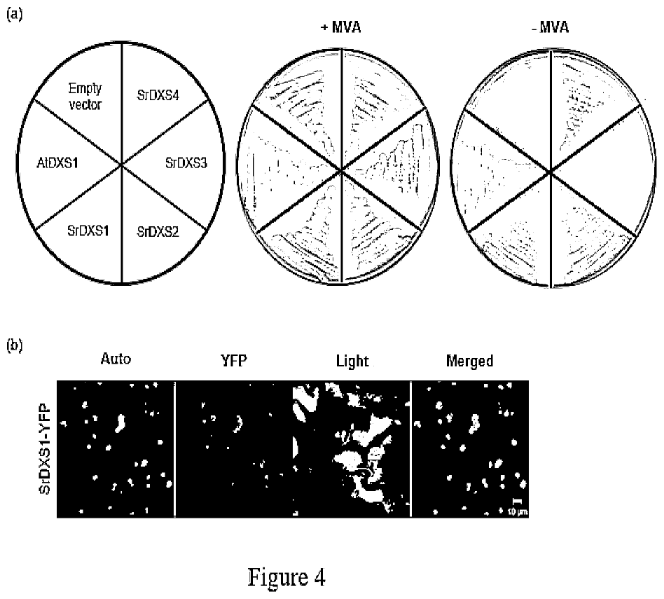

Four Stevia DXS homologs (SrDXS1-4) were identified from the RNA-seq data of Stevia leaves (Kim et al., 2015). To investigate if all four SrDXSs were functionally active, a complementation assay was carried out using a dxs-deficient Escherichia coli. FIG. 4a shows that dxs⁻ E. coli transformed with all SrDXSs except SrDXS3 were able to grow on selection media, similar to the Arabidopsis DXS1 (AtDXS1) positive control, indicating their functionality. Among the 4 SrDXS homologs, only SrDXS1 was suggested to be involved in SG biosynthesis based on the correlation between its expression pattern and the site of SGs biosynthesis (Kim et al., 2015). Transient expression of the yellow fluorescent protein (YFP) fused-SrDXS1 in Nicotiana benthamiana leaves showed that it localizes to the chloroplast (FIG. 4b). Therefore, SrDXS1 was selected for Stevia transformation.

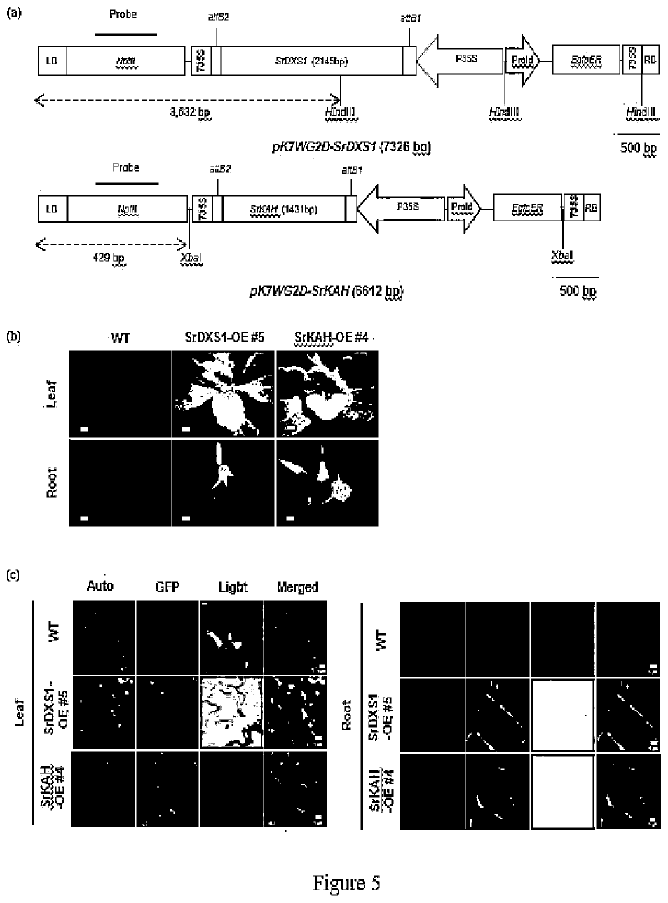

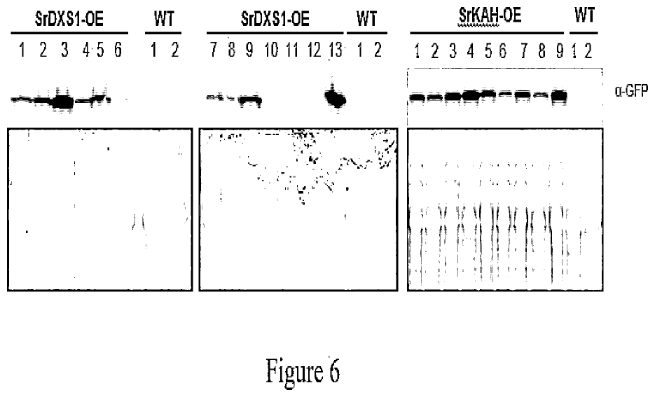

Next, the full-length ORFs of SrDXS1 and SrKAH were cloned into the pK7WG2D vector under the control of the cauliflower mosaic virus (CaMV) 35S promoter for Stevia transformation (FIG. 5a). Using transformation protocol described herein, 13 and 9 lines of transgenic Stevia plants were produced overexpressing SrDXS1 (SrDXS1-OE) and SrKAH (SrKAH-OE), respectively. The GFP visual marker enabled the efficient selection of transgenic Stevia plants emitting GFP signals from leaf and root tissues of SrDXS1-OE and SrKAH-OE lines under a fluorescence stereomicroscope and confocal laser scanning microscope (CLSM; FIGS. 5b and 5c). GFP expressions in leaves of each transgenic Stevia lines were also confirmed by immunoblot analysis (FIG. 6).

### Example 5

### Analysis of Transgenic Stevia Lines

To verify if exogenous SrDXS1 or SrKAH was integrated into the Stevia genome, genomic PCR analysis of the transgene from each transgenic line was performed. Genomic DNA amplification corresponding to the expected size of each transgene was observed for all the SrDXS1-OE or SrKAH-OE lines and the respective positive control lanes, but not for wild type (WT; FIGS. 7a and 7b).

After confirming the existence of full-length ORFs of each transgene in transgenic Stevia plants, digoxygenin (DIG)-based Southern blot analysis was performed to determine transgene copy number for each line with nptII-specific probe (FIG. 5a). FIGS. 7c and 7d show that all SrDXS1-OE and SrKAH-OE lines contained one or more copies of the transgene, demonstrating stable transgene integration into the Stevia genome. No bands were detected in the two WT lanes.

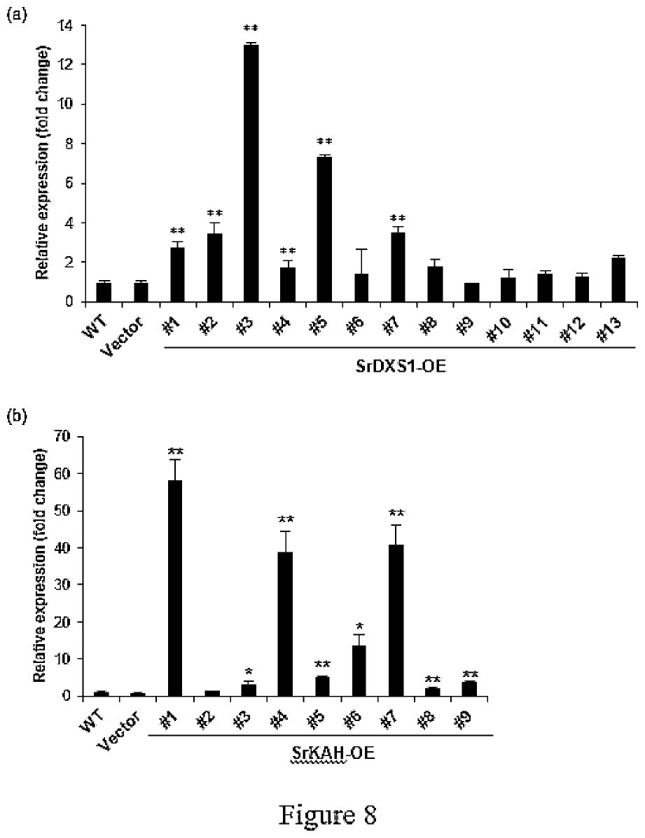

Then, the expression levels of SrDXS1 and SrKAH was analyzed in SrDXS1-OE and SrKAH-OE lines, respectively. FIG. 8a shows an approximately 1.5 to 13 fold increase in the expression levels of SrDXS1 among the transgenic lines compared to control. However, the expression levels of SrDXS1 in SrDXS1-OE lines did not correlate with the transgene copy number. Among the top 5 SrDXS1-OE lines, four of them had a single transgene inserted into their genome (FIGS. 7c and 8a). For further analysis, the single copy lines, SrDXS1-OE #1, #3 and #5 with different levels of SrDXS1 overexpression were chosen.

Among SrKAH-OE lines that contained single copy transgene, lines #1, #4 and #7 showed around 40-60 fold higher expression of SrKAH compared to that of WT while line #2 did not show SrKAH overexpression, and line #9 only had a small increase of around four-fold (FIG. 8b). For further analysis of the effects of SrKAH overexpression, we selected lines #1, #4, and #9 with varying expression levels were selected, and line #2 was included as an internal control.

### Example 6

### Steviol Glycosides (SGs) Content Increased in Transgenic Stevia Plants

It is known that Stevia is a self-incompatible plant and its self-pollination result in sterile seed set (Raina et al., 2013). Under local environmental conditions, harvesting viable transgenic T1 seeds was also unsuccessful. Therefore, the in vitro transgenic lines were propagated by cutting method and monitored the GFP signals emitted. Transgenic Stevia plants showing GFP expression in whole tissues were transferred into the soil for hardening and grown in the greenhouse for three weeks before analysis. Using this method, each transgenic line maintained for further analysis and obtain reproducible results.

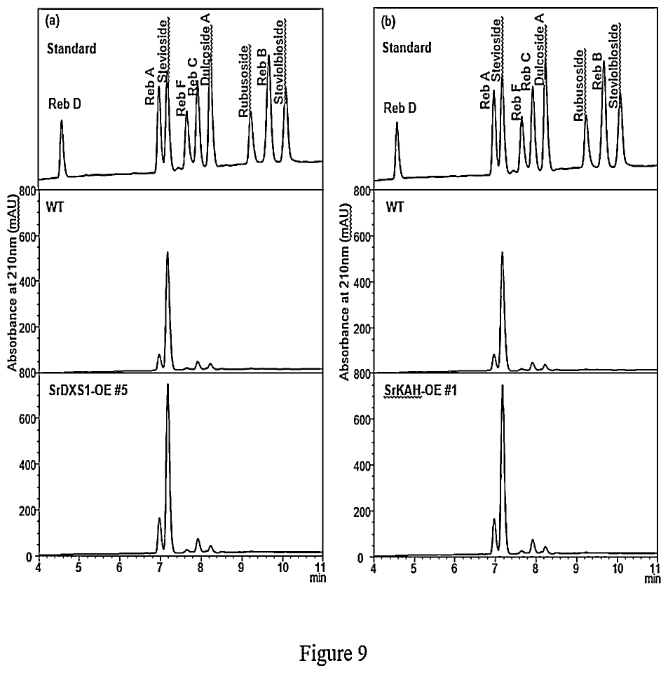

In order to investigate the effect of SrDXS1 or SrKAH overexpression on SGs production, the leaf extracts of the transgenic lines were analyzed. As leaf SGs content can differ according to their nodal position, leaves from the same position of each line were harvested. Each SG peak was identified by comparing their retention time with that of their authentic standards (FIGS. 9a and 9b).

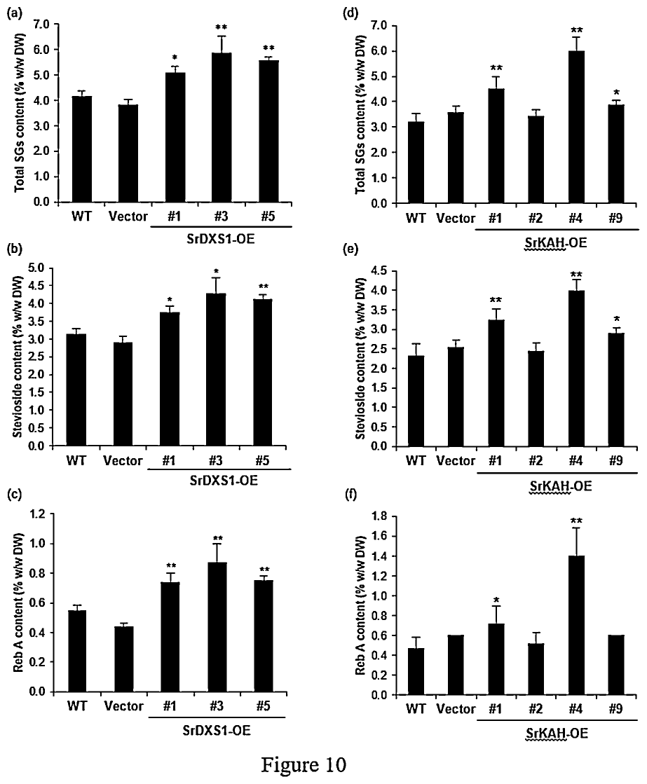

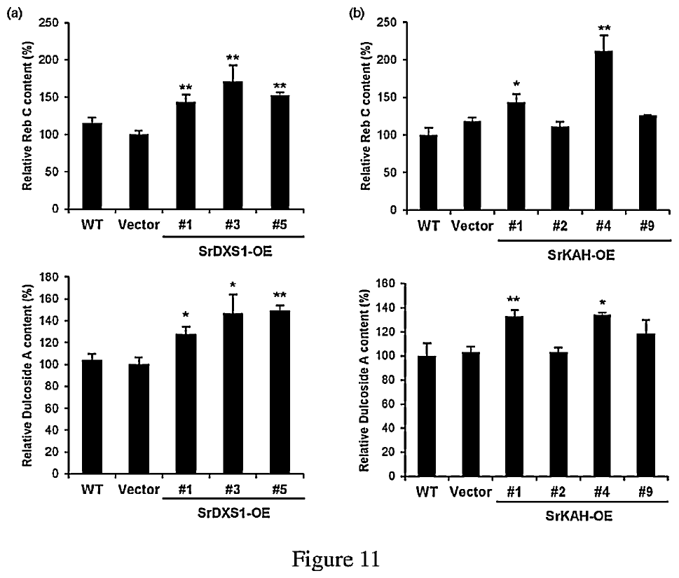

By summing up the concentration of the top 4 most abundant SGs (stevioside, Reb A, Reb C and dulcoside A) in each of the SrDXS1-OE lines, an increase in SGs content in the transgenic lines as compared to the controls (FIG. 10a) was found. The total SGs content was the highest in SrDXS1-OE line #3 at 5.9% (w/w dry weight, DW), followed by 5.6% (w/w DW) in line #5 and lastly 5.1% (w/w DW) in line #1 (FIG. 10a), in agreement with their relative SrDXS1 expression levels (FIG. 8a). These total SGs content in the transgenic lines represent an increase of between 33%-54% and 23%-42% compared to the 3.8% (w/w DW) and 4.1% (w/w DW) total SGs content in the vector-only control line and WT, respectively (FIG. 10a). Stevioside, which is the most abundant SG in Stevia, had concentrations of between 3.7%-4.3% (w/w DW) in the overexpression lines, increasing up to 20%-47% compared to controls (FIG. 10b). Similar patterns of SGs increase for Reb A, Reb C and dulcoside A were found in SrDXS1-OE lines (FIG. 10c and FIG. 11a). These results suggest that the overexpression of SrDXS1 in Stevia leads to a proportional increase in each SG.

In the SrKAH-OE lines, the total amount of SGs was able to reach up to 88% higher than that of WT (FIG. 10d). Corresponding to their expression levels, SrKAH-OE lines #1 and #4 accumulated the highest total amount of SGs at 4.5% (w/w DW) and 6% (w/w DW), respectively (FIGS. 8b and 10d). On the other hand, SrKAH-OE #9 with only a four-fold increase in SrKAH transcript had total SGs content of 3.9% (w/w DW), indicating a moderate increase of 8%-22% from the controls (FIGS. 8b and 10d). SrKAH-OE line #2, an internal control line that shows similar expression levels of SrKAH with WT, did not contain higher total SGs content, confirming that elevated SrKAH transcript levels resulted in higher SGs in transgenic Stevia plants (FIG. 10d). Taking a closer inspection at the individual SGs, stevioside was present in concentrations of up to 4% (w/w DW) among the overexpression lines, which was an increase of 57%-71% compared to controls (FIG. 10e). A dramatic 133%-200% increase in Reb A content compared to controls was observed in SrKAH-OE #4 (FIG. 10f). In addition, statistically significant increases of Reb C and dulcoside A content were also found in the two SrKAH high expressers, SrKAH-OE lines #1 and #4, having a similar pattern of increase with total SGs in SrDXS1-OE lines (FIG. 11b).

### Example 7

### Phenotype of Transgenic Stevia Plants

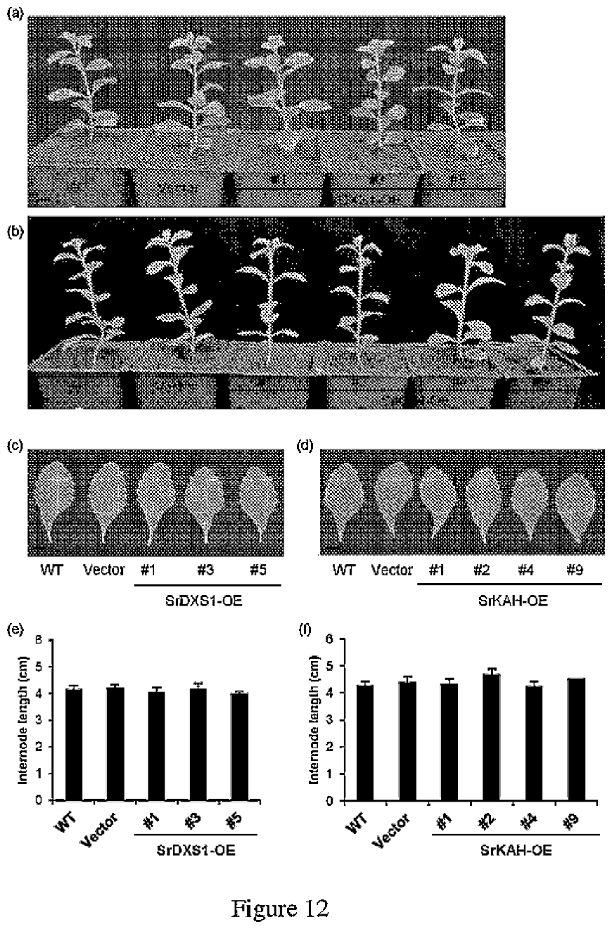

GA is known to be involved in plant growth and development and its reduction results in phenotypic changes such as dwarfism, reduced internode length, and small dark leaves (Carrera et al., 2000; Thomas and Sun, 2004). Because GA is synthesized through the MEP pathway, the phenotypes of SrDXS1-OE lines were observed. FIGS. 12a-12f show that SrDXS1-OE lines did not show any morphological difference from controls. The height of the plants, size of the leaves and the internode length among the two month-old Stevia plants were comparable (FIGS. 12a, 12c and 12e). This suggests that GA levels in the transgenic lines might not be affected significantly by SrDXS1 overexpression. The effects of SrKAH overexpression on GAs biosynthesis was also examined, since this overexpression may divert kaurenoic acid from the GA production, leading to GA deficiency. FIGS. 12b and 12d show that SrKAH-OE lines did not exhibit any symptoms of dwarfism. The leaf size and color and internode length were indistinguishable from controls.

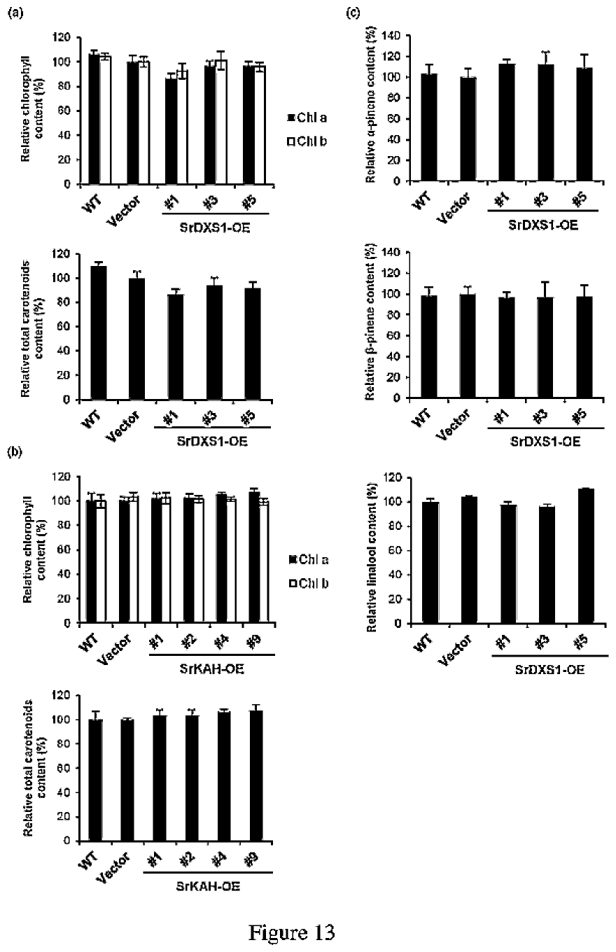

Other than GA, the relative concentration of chlorophyll a, chlorophyll b and total carotenoids was also determined because these compounds are also derived from the MEP pathway (Rodriguez-Concepcion and Boronat, 2002). FIGS. 13a and 13b shows that there were no significant changes in chlorophylls and carotenoids content in both SrDXS1-OE and SrKAH-OE lines. Additionally, the concentration of a few monoterpenes that were present in the Stevia leaf tissues were measured since monoterpenes can also be synthesized from the MEP pathway (Kim et al., 2015). Using GC-MS analysis, the relative amount of linalool, α-pinene and β-pinene were determined (FIG. 13c). There were no statistically significant changes to the amount of monoterpenes in the leaves of SrDXS1-OE lines compared to those of controls. Hence, the results show that SrDXS1 and SrKAH overexpression could both increase SGs content in transgenic Stevia without changing the abundance of other metabolites or having any detrimental effects on their growth and development.

### Example 8

### Materials and Methods for Examples 9-13

Stevia transformation: The full-length ORF of SrUGT76G1 (GenBank Accession number, AY345974; Richman et al., 2015) that was PCR-amplified from the cDNA of Stevia leaves using primers listed in Table 5 was cloned into the pK7WG2D vector using GATEWAY technology (Invitrogen). After confirmation by sequencing, the expression vector was transformed into the Agrobacterium strain AGL2. Transformation of Stevia using this Agrobacterium is described above. Briefly, the leaf explants were co-cultivated with Agrobacterium on co-cultivation media (0.25 mg/L 2,4-dichlorophenoxyacetic acid (2,4-D)+100 μM acetosyringone) for 3 days and transferred onto callus induction media (1 mg/L 6-benzylaminopurine (BA)+0.5 mg/L 3-indoleacetic acid (IAA)+125 mg/L cefotaxime+50 mg/L kanamycin) after washing. After 3-4 weeks of incubation, transformed calli that emitted GFP signals under a fluorescent microscope were further transferred onto shoot induction media (2 mg/L BA+0.25 mg/L IAA+125 mg/L cefotaxime+50 mg/L kanamycin) and subcultured every 3-4 weeks. The explants were incubated at 25° C. in the dark throughout. Regenerated shoots with GFP signals were then transferred onto rooting media (0.5 mg/L IAA+125 mg/L cefotaxime) under long day (LD) condition (16 h Light/8 h Dark). Fully developed transgenic plants were propagated in vitro by cutting method and transferred onto soil after roots developed. For hardening, plants were placed in a plant growth chamber at 25° C. with exposure to LD condition and covered with a transparent plastic dome. Subsequently, plants were shifted to the greenhouse and subjected to the local climate conditions.

TABLE 5
Primers

| | Forward sequence (F) | Reverse sequence (R) |
|---|---|---|
| Name | (SEQ ID NO:) | (SEQ ID NO:) |
| | For RT-PCR | |
| SrDXS1 | GCAACACTGTCGGAGAGAGGTG (35) | CTGTTAACTCCACCACACCAAGAC (36) |
| SrDXR1 | TCCTGAAGGTGCTTTGAGGCGT (37) | GACCCGTAAAGATAATGAGCTTCG (38) |
| SrMCT | AGATGCCAGAGATAACATCAGTGTG (39) | ATGCTCCAACTCGCAACCCATCA (40) |
| SrCMK | CAGGCCGAGGTGAGATTGTTCA (41) | CAGGCGGTTCCAAATCATTTACAC (42) |
| SrMDS | GCTGCGAAGCTCACTCTGATGGTG (43) | CAGCTTCATGCATCAATCTCACTG (44) |
| SrHDS | AGGCACACGTTTGGTGGTATCTT (45) | GAAAGTTATGTGGTGAAGAACAGG (46) |
| SrHDR | CATCCTTGGTGGTAAGCTTAACGG (47) | CTACTCCATATTTACTCATCATGGTTC (48) |
| SrGGDPS3 | CATGGGTTCACTCATGCTCCATGT (49) | TGAAGCTGGATTCCTGGATCTC (50) |
| SrCPS | TTCCGGTGTAAAGCGGTATC (51) | CATTGCTTTCACGCTCTCAA (52) |
| SrKS1 | TCCGGCTTTCTATGGTTGAC (53) | AACCGAAAGGCTAAAGCACA (54) |
| SrKO1 | TCGATTAAAACCGGAGCAAC (55) | CCCAAAACAGCGGTCAGTAT (56) |
| SrKAH | GAGCAACTAGAGATATCGAAGACG (57) | CACTCCAGTGTAGCTTCCATCCT (58) |
| SrUGT85C2 | GTCATTGAGGTATAATCACATTTACACC (59) | TCACCAAGTTTGATCGGATGATCC (60) |
| SrUGT74G1 | GAAATCACCACACGTTCTACTCATC (61) | GAGGTGGTGGTGGTGTTACTGTG (62) |
| SrUGT76G1 | TATTCCCGGTACCATTTCAAGGC (63) | CGGTAGATTGGAAATGCGTTCGTC (64) |
| SrActin | TCTTGATCTTGCTGGTCGTG (65) | GAGCAAGAACTTGAAACCGC (66) |
| | For Gateway cloning | |
| SrUGT76G1 | AAAAAGCAGGCTTCATGGAAAATAA | AGAAAGCTGGGTGTTACAACGATGAAA |
| | AACGGAGACCA (67) | TGTAAGAAACT (68) |
| | For Southern blot probes and confirmatory PCR | |
| | in transgenic lines | |
| CaMV 35S | TAGAGAGGCCTACGCGGCAGGT (69) | GTCATCCCTTACGTCAGTGGAGAT (70) |
| CaMV 35S-seq F | ATCTCCACTGACGTAAGGGATGAC (71) | |
| SrUGT76G1-CR | | TTACAACGATGAAATGTAAGAAACTAGA (72) |

Verification of transgenic Stevia plants by genomic PCR and Southern blot analysis: Cetyltrimethylammonium bromide (CTAB)-based extraction method was used to extract genomic DNA (gDNA) from Stevia leaves (Rogers and Bendich, 1989).

For genomic PCR, approximately 100 ng of gDNA was added to a PCR reaction mix containing forward primers specific to the CaMV 35S promoter and reverse primers specific to the 3′ end of SrUGT76G1 (Table 5).

Southern blot analysis was carried out using a digoxygenin (DIG)-labelled probe specific to the full length of the CaMV 35S promoter. gDNA extracted from the SrUGT76G1-OE lines were digested with HindIII and resolved on a 0.8% agarose gel together with the DIG-labelled DNA molecular weight marker II (Roche). The agarose gel was then treated for the transfer of fragmented gDNA onto a positively charged nylon membrane (Hybond-N+) as mentioned previously (Zheng et al., 2018). Following DIG-based Southern blot hybridization (Roche), chemiluminescence from the membrane was detected using the ChemiDoc Touch Imaging System (Bio-Rad).

Expression analysis by quantitative real-time PCR (qRT-PCR): Total RNA was extracted from homogenized Stevia leaves using TRIzol reagent (Invitrogen) and contamination from DNA was removed with deoxyribonuclease I (DNaseI; Roche). For cDNA synthesis, 1 μg of total RNA was used with M-MLV Superscript II (Promega). To determine the transcript abundance of SrUGT76G1 and all other genes in the SGs biosynthesis pathway, qRT-PCR was performed using SYBR Premix Ex Taq II (Takara) and quantified on Applied Biosystems (USA) 7900HT fast real-time PCR system. Primers used are listed in Table 5. Primer specificity was verified by sequencing of product from regular PCR and melting curve analysis. The abundance of Stevia actin transcript was used as an internal control for normalization.

Steviol glycosides content analysis by High-Performance Liquid Chromatography: Leaves for SGs content analysis were harvested from the 6^{th} node of plants grown in the greenhouse for 3 weeks. After drying the leaves overnight in a 60° C. oven, the samples were ground and 30 mg of the powdered leaves were extracted using 3 mL of water twice in an ultrasonic bath maintained at 50° C. for 20 min. The extracts were centrifuged at 3000 rpm for 15 min. 1 mL of supernatant filtered through a 0.45 μm filter was loaded onto a solid phase extraction (SPE) column C2 (Agilent) and washed with acetonitrile:water (20:80, v/v) before elution in 1 mL of methanol:acetonitrile (50:50, v/v). To analyze SGs content, 5 μL of the eluted sample was applied on a Shimadzu Nexera X2 ultra-high performance liquid chromatography (UHPLC) fitted with a Shim-pack VP-ODS column (250×4.6 mm, i.d. 5μm) and detected by a photodiode array detector (SPD-M30A with high sensitivity cell). The elution was performed over 24 min with a 30-80% acetonitrile gradient at a flow rate of 1.0 ml/min. Column oven was maintained at 40° C. Chromatogram detected at a wavelength of 210 nm was used for SGs identification and quantification. Peak assignment was based on comparison with elution profile of known standards (ChromaDex) and the concentration of each SG was determined from the standard curves of the respective SGs.

Chlorophylls and total carotenoids analysis: For the measurement of chlorophylls and total carotenoid content, leaves were harvested from the 4th and 5th nodes of plants that were grown in the greenhouse for 3 weeks and frozen in liquid nitrogen. After homogenization, 200 mg of the leaves were extracted twice with 2 ml of 100% methanol at room temperature for 1 h with constant shaking in the dark. The extracts were pooled and diluted 5 folds before analysis on an Infinite M2000 microplate reader (Tecan). Absorbance values at 3 different wavelengths, 666 nm, 653 nm and 470 nm, were used to calculate the relative amount of chlorophyll a, chlorophyll b and total carotenoids present in the leaves based on previously reported formula (Lichtenthaler and Wellburn, 1983).

Expression of recombinant UGT76G1 and UDP-glucosyltransferase activity assay: The full-length cDNA of SrUGT76G1 was cloned into pDEST15 to obtain GST-tag fused protein. The resulting expression vector was transformed into E. Coli BL21 (DE3)-derived Rosetta strain (Novagen) and grown under appropriate antibiotics. GST-tagged SrUGT76G1 recombinant protein was purified by glutathione agarose beads (ThermoFisher Scientific). About 1 μg of recombinant protein was used for enzyme assay with 50 μM of the substrate (dulcoside A or Reb A) in an assay buffer (50mM HEPES, pH 7.5, 3mM MgCl₂, 10 μg/m1 Bovine Serum Albumin). To initiate the reaction, a 1 mM UDP-glucose mixture (997.5 μM UDP-glucose and 2.25 μM UDP-[¹⁴C]-glucose, 2.78 kBq, Amersham Biosciences) was added. In vitro glucosyltransferase activity assays were performed as described by Richman et al. (2005). The assay was carried out at 30° C. for 2 h and extracted twice with 100 μl of water-saturated 1-butanol. Pooled fractions were dried in a vacuum centrifuge and resuspended in 10 μl water-saturated 1-butanol for thin layer chromatography (TLC) analysis. The TLC was performed with 10 μl of reaction products using chloroform: methanol: water (15:10:2 v/v/v) as the mobile phase on a silica gel-coated TLC plate (Fluka) in a mobile phase saturated glass chamber. SGs standards were run under the same condition. After air-drying, the image on the TLC plate was captured on a storage phosphor screen in a phosphorimager cassette (Bio-Rad) for 2-3 d and visualized on a Typhoon 9200 imager (Amersham Biosciences).

HPLC analysis of in vitro glucosyltransferase activity assay mixture: In vitro glucosyltransferase activity assay was performed as indicated in the TLC analysis, but 5 mM of UDP-glucose was used without UDP-[¹⁴C]-glucose and the incubation time was increased to 16 h. Samples were extracted 3 times with water-saturated 1-butanol and dried completely in a vacuum centrifuge. Dried samples were dissolved in MeOH for UHPLC analysis in accordance with the method mentioned for SGs content analysis.

### Example 9

### Transgenic Stevia Plants Overexpressing SrUGT76G1

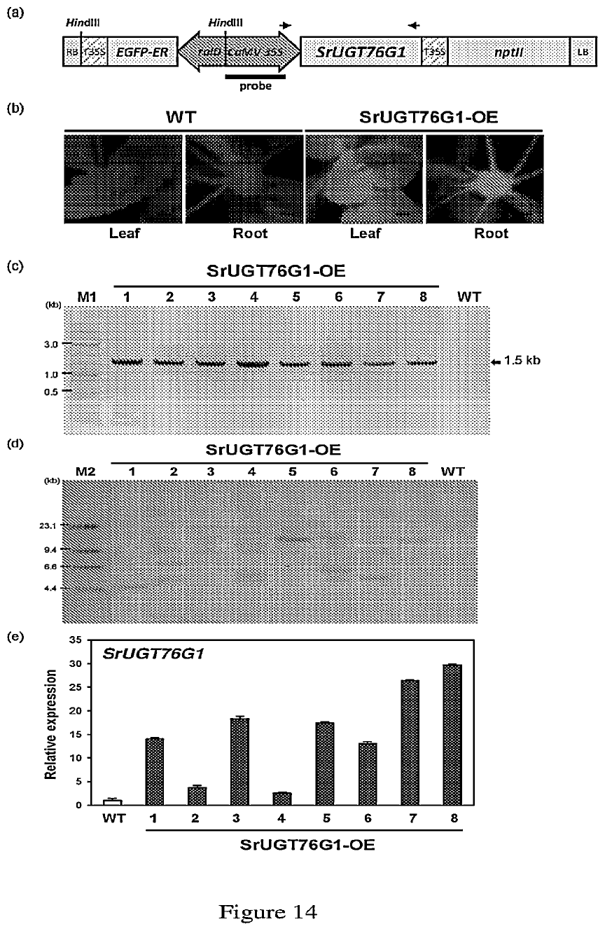

Since SrUGT76G1 has been known to be involved in the conversion of stevioside to Reb A, steviobioside to Reb B, and Reb D to Reb M (Richman et al., 2005; Olsson et al., 2016), it was hypothesized that its overexpression could increase or alter the proportion of these SGs. Therefore, the full-length open reading frame (ORF) of SrUGT76G1 was cloned into pK7WG2D under the control of the cauliflower mosaic virus (CaMV 35S) promoter for the Agrobacterium-mediated transformation of Stevia (FIG. 14a). Using the transformation method of Stevia described herein that employs green fluorescent protein (GFP) as a visual marker (Zheng et al., 2018), eight transgenic lines emitting GFP signals were generated (FIG. 14b).

To verify the integration of the exogenous SrUGT76G1 in transgenic Stevia, a genomic PCR analysis on the genomic DNA extracted from each SrUGT76G1-overexpressing lines (SrUGT76G1-OE) was carried out. A band corresponding to the expected size of the SrUGT76G1 transgene was detected in all the transgenic lines except the WT (FIG. 14c). For further investigation into the copies of transgene present in each line, a digoxygenin (DIG)-based Southern blot analysis was then performed on HindIII-digested genomic DNA extracted from each line using a CaMV 35S promoter-specific probe. FIG. 14d shows that only line #8 contained a single copy of transgene while lines #1 and #5 had two copies of the transgene and the remaining lines had three or more copies of the transgene integrated.

Next, the transcript levels of SrUGT76G1 in the SrUGT76G1-OE lines were analyzed using qRT-PCR. FIG. 14e shows that the transcript levels of SrUGT76G1 were approximately 3 to 30 folds higher in the SrUGT76G1-OE lines as compared to WT, with lines #8 and #4 being the highest and lowest expressers, respectively. Therefore, four lines, lines #1, #5, #7 and #8, which showed higher expression of the SrUGT76G1, were selected for further analysis on the effect of SGs abundance and/or changes in SGs ratio.

### Example 10

### Alteration of Steviol Glycosides Composition in Transgenic Stevia Plants

To measure the SGs content in SrUGT76G1-OE lines, were multiplied through in vitro cutting propagation and harvested leaves from the same nodal position after hardening in the soil. Extracted SGs from the dried leaves were analyzed using high-performance liquid chromatography (HPLC) and individual SGs were identified by the alignment of their retention time with that of authentic standards (FIG. 15). Intriguingly, by comparing the representative chromatograms of each transgenic line to that of WT, it was found that a noticeable change in the relative abundance of Reb A to stevioside (FIG. 15). In all SrUGT76G1-OE lines except #1, the Reb A peak even surpassed the peak for stevioside (FIG. 15). However, any additional SGs that were previously mentioned to be products of in vitro assays involving SrUGT76G1 such as Reb B and Reb M could not be detected.

For a more detailed study on the SGs content, the peaks of the top four most abundant SGs present in the leaves were quantified. By summing up the four SGs, no significant difference could be seen in the total SGs content in the SrUGT76G1-OE lines, which were between 3.56-4.04% (w/w DW), compared to the 3.70% (w/w DW) in WT (FIG. 16a). However, significant changes were observed in the individual SGs, especially for stevioside and Reb A, in the SrUGT76G1-OE lines (FIGS. 16b and 16c). Compared to WT which has stevioside content of 2.71% (w/w DW), the transgenic lines showed content that were between 25-61% lower. For instance, the transgenic line with the lowest stevioside content, line #8, had concentrations of only 1.07% (w/w DW). Even line #1 that possessed the highest stevioside content among the transgenic lines at 2.04% (w/w DW), was still 25% lower than that of WT (FIG. 16b). It should be noted that the reduction of stevioside in transgenic lines correlated negatively with SrUGT76G1 expression levels (FIGS. 16b and 14e).

On the other hand, Reb A content in the SrUGT76G1-OE lines was significantly increased by up to 137.3% compared to WT (FIG. 16c). In WT, the Reb A content was 0.79% (w/w DW), but in lines #1 and #5 that had the lowest and highest Reb A content, this was increased to 1.26% (w/w DW) and 1.87% (w/w DW), respectively (FIG. 16c). To quantify the relative increase in Reb A to stevioside in the transgenic lines, the ratio of Reb A to stevioside (Reb A/stevioside ratio) was calculated and a remarkable improvement in this ratio compared to WT was observed. In WT, the Reb A/stevioside ratio was 0.30 and this increased to 0.62, 1.04, 1.25 and 1.55 in the SrUGT76G1-OE lines #1, #7, #5 and #8, respectively (FIG. 16d). The higher Reb A/stevioside ratio was positively correlated with the transcript levels of SrUGT76G1 in the SrUGT76G1-OE lines (FIGS. 14e and 16d). Among the SrUGT76G1-OE lines, line #8 had both the greatest Reb A/stevioside ratio and the highest SrUGT76G1 expression levels, while line #1 showed the opposite (FIGS. 14e and 16d). These results demonstrate for the first time that SrUGT76G1 could indeed convert stevioside to Reb A in planta as well.

Other than changes in Reb A/stevioside ratio, the proportion of Reb C to dulcoside A was also affected. The dulcoside A concentration in the SrUGT76G1-OE lines were between 13.2-38.0% lower than that in the WT (FIG. 17a). On the other hand, Reb C content was increased by between 17.2-37.8% in the transgenic lines compared to WT (FIG. 17b). These results imply that SrUGT76G1 might be involved in the conversion of dulcoside A to Reb C in Stevia.

### Example 11

### Phenotypes of Stevia with SrUGT76G1 Overexpression

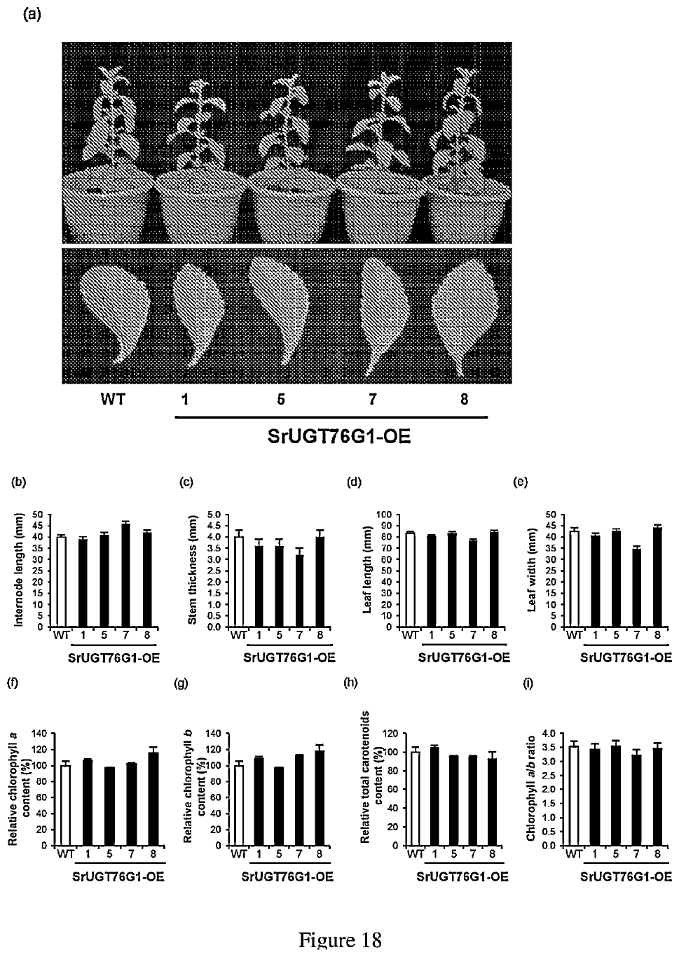

Plants exhibit phenotypic changes such as dwarfism and reduced internode length under reduced GA content (Thomas and Sun, 2004). It has been reported that transient knockdown of the SrUGT76G1 in Stevia led to an increase in GA levels so the overexpression of SrUGT76G1 may have an opposite effect (Guleria and Yadav, 2013). Hence, the growth and development of the transgenic Stevia plants were monitored. FIG. 18a shows that there were no obvious differences in morphology between the transgenic lines and WT. Internode length measurements made at 8 weeks after the transfer into the soil were comparable between the WT and overexpression lines at 40 mm and between 39-46 mm, respectively (FIG. 18b). In addition, the stem thickness and leaf size of the SrUGT76G1-OE lines were also very similar to those of WT (FIGS. 18c-18e).

Chlorophylls and total carotenoids content, which are essential metabolites that share some precursors with SGs biosynthesis (Rodriguez-Concepcion and Boronat, 2002) were quantified. Similarly, the content of these metabolites in the Stevia with SrUGT76G1 overexpression did not differ from WT (FIGS. 18f-18h). Additionally, the chlorophyll a/b ratios were also comparable to that of WT indicating that the photosynthetic capacity of the transgenic lines was very similar to WT (FIG. 18i). Therefore, other than changes in the Reb A/stevioside ratio, any other abnormalities in SrUGT76G1-OE lines compared to WT Stevia plant were not found.

### Example 12

### Expression Pattern of Other SGs Pathway Genes

To investigate if the overexpression of SrUGT76G1 somehow triggers a feedback loop that affects the expression of other genes in the SGs biosynthesis pathway, the transcript levels of the genes involved in the synthesis of the steviol precursor were measured. FIGS. 19a and 19b shows that the expression of all gene in the MEP pathway including SrDXS1, SrDXR1, SrCMS, SrCMK, SrMCS, SrHDS, SrHDR and SrGGDPS3, as well as the downstream genes for isoprenoid biosynthesis, SrCPS1, SrKS1, SrKO1 and SrKAH, were not notably up- or down-regulated in the SrUGT76G1-OE lines compared to WT. These results could possibly explain the minimal changes in total SGs content observed in the transgenic lines (FIG. 16a). Similarly, changes in the transcript abundance for two other SrUGTs, SrUGT85C2 and SrUGT74G1, were almost negligible in the SrUGT76G1-OE lines (FIG. 19c). This corresponds to an analysis showing no differences in the amount of rubusoside, which can be synthesized by the combined activities of SrUGT85C2 and SrUGT74G1 on steviol (FIG. 15; Humphrey et al., 2006).

### Example 13

### An Additional Function of SrUGT76G1

In addition to changes in Reb A/stevioside ratio, the Reb C/dulcoside A ratio in the SrUGT76G1-OE lines was also affected (FIG. 17b). SrUGT76G1 has so far been shown to be involved in 1,3-glucosylations of C₁₃- and C₁₉-positioned glucose of eight different SG in vitro (Olsson et al., 2016). However, the potential conversion of dulcoside A to Reb C by the 1,3-glucosylation activity of SrUGT76G1 has not yet been demonstrated.

To determine if SrUGT76G1 has an additional function for Reb C production from dulcoside A, in vitro assays using recombinant SrUGT76G1 protein with UDP-glucose as the sugar donor and dulcoside A as the acceptor were performed. Thin-layer chromatography (TLC) analysis shows that the glutathione S-transferase (GST)-fused SrUGT76G1 (GST-SrUGT76G1) recombinant protein, but not GST alone, was able to produce Reb C from dulcoside A in the reaction mixture (FIG. 20a). In the positive control using stevioside as the acceptor, Reb A was obtained as expected (FIG. 20a).

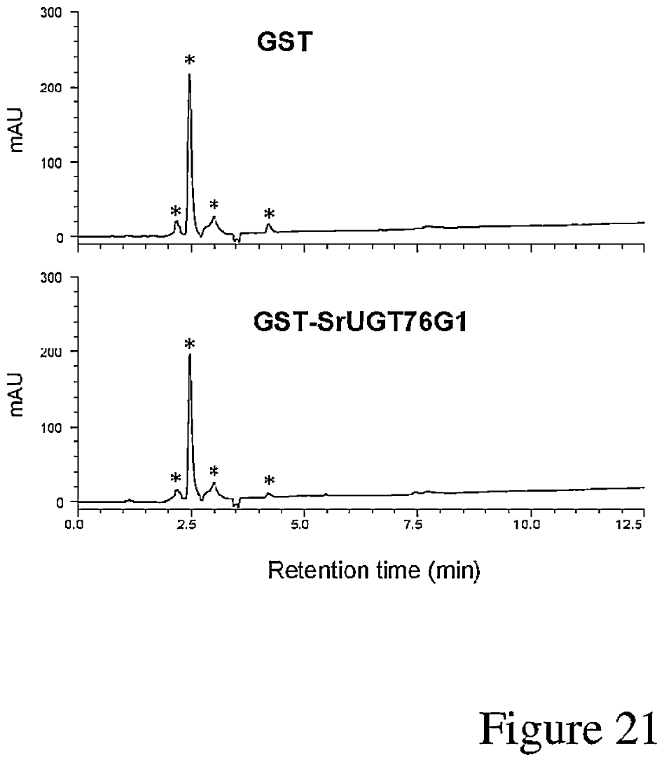

This reaction was further verified by HPLC analysis. The negative control assay is shown in FIG. 21. In the positive control assay, Reb A was produced from the reaction mix containing GST-UGT76G1 with stevioside (FIG. 20b). An additional peak for Reb I, which can be converted from Reb A by GST-UGT76G1, was detected as well (FIG. 20b; Olsson et al., 2016). Most importantly, Reb C was detected in the reaction mixture with dulcoside A once again, confirming the TLC analysis. This result demonstrates that in addition to SGs acceptors reported so far, SrUGT76G1 also performs 1,3-glucosylation on the C₁₃-positioned glucose on dulcoside A to form Reb C (FIG. 20c). Moreover, it was confirmed in planta by the increased Reb C content and a concurrent decreased dulcoside A content observed in the SrUGT76G1-OE lines. Interestingly, we detected another reaction product was detected in the HPLC analysis with a retention time that does not correspond to any of our standards (FIG. 20b). Since SrUGT76G1 catalyzes 1,3-glucosylation of C₁₃- or C₁₉-positioned glucose of SG, it was postulated that this novel peak is likely to be produced in vitro from the 1,3-glucosylation of the C₁₉-positioned glucose on dulcoside A (FIG. 20c).

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Aman, N., Hadi, F., Khalil, S. A., Zamir, R. and Ahmad, N. (2013) Efficient regeneration for enhanced steviol glycosides production in Stevia rebaudiana (Bertoni). C R Biol 336, 486-492.

Anbazhagan, M., Kalpana, M., Rajendran, R., Natarajan, V. and Dhanavel, D. (2010) In vitro production of Stevia rebaudiana Bertoni. Emirates Journal of Food and Agriculture 22, 216.

Ara, K., Leland J., C. and Peter B., K. (2009) The use of plant cell biotechnology for the production of phytochemicals In: Recent Advances in Plant Biotechnology (Ara, K. and Peter, B. K. eds), pp. 15-33. Springer Science & Business Media.

Bespalhok-Filho, J.C. and Hattori, K. (1997) Embryogenic callus formation and histological studies from Stevia rebaudiana (Bert.) Bertoni floret explants. Revista Brasileira de Fisiologia Vegetal 9, 185-188.

Bevan, M. W., Flavell, R. B. and Chilton, M. D. (1983) A chimaeric antibiotic resistance gene as a selectable marker for plant cell transformation. Nature 304, 184-187.

Bowles, D., Isayenkova, J., Lim, E. K., Poppenberger, B. (2005) Glycosyltransferases: managers of small molecules. Current Opinion in Plant Biology 8: 254-263.

Brandle, J. E. and Telmer, P. G. (2007) Steviol glycoside biosynthesis. Phytochemistry 68, 1855-1863.

Carrera, E., Bou, J., García-Martínez, J. L. and Prat, S. (2000) Changes in GA 20-oxidase gene expression strongly affect stem length, tuber induction and tuber yield of potato plants. The Plant Journal 22, 247-256.

Ceunen, S. and Geuns, J. M. C. (2013) Steviol Glycosides: Chemical Diversity, Metabolism, and Function. Journal of Natural Products 76, 1201-1228.

Ceunen, S., Wim de, B., Compernolle, F., Mai, A. H. and Geuns, J. M. (2013) Diterpene glycosides from Stevia phlebophylla A. Gray. Carbohydrate research 379, 1-6.

Chen, J., Hou, K., Qin, P., Liu, H., Yi, B., Yang, W. and Wu, W. (2014) RNA-Seq for gene identification and transcript profiling of three Stevia rebaudiana genotypes. BMC Genomics 15, 571.

Christensen, A. H. and Quail, P. H, (1989). Sequence analysis and transcriptional regulation by heat shock of polyubiquitin transcripts from maize. Plant Mol Biol 12:619-632.

Christensen, A. H. et al. (1992). Maize polyubiquitin genes: structure, thermal perturbation of expression and transcript splicing, and promoter activity following transfer to protoplasts by electroporation. Plant Mol Biol 18:675-689.

Cordoba, E., Salmi, M. and León, P. (2009) Unravelling the regulatory mechanisms that modulate the MEP pathway in higher plants. Journal of Experimental Botany 60, 2933-2943.

Corredoira, E., Valladares, S., Allona, I., Aragoncillo, C., Vieitez, A. M. and Ballester, A. (2012) Genetic transformation of European chestnut somatic embryos with a native thaumatin-like protein (CsTL1) gene isolated from Castanea sativa seeds. Tree Physiology 32, 1389-1402.

Das, A. and Mandal, N. (2010) Enhanced development of embryogenic callus in Stevia rebaudiana Bert. by additive and amino acids. Biotechnology 9, 368-372.

Duran-Vila, N., Gogorcena, Y., Ortega, V., Ortiz, J. and Navarro, L. (1992) Morphogenesis and tissue culture of sweet orange (Citrus sinensis (L.) Osb.): Effect of temperature and photosynthetic radiation. Plant Cell, Tissue and Organ Culture 29, 11-18.

Estévez, J. M., Cantero, A., Reindl, A., Reichler, S. and León, P. (2001) 1-Deoxy-d-xylulose-5-phosphate Synthase, a Limiting Enzyme for Plastidic Isoprenoid Biosynthesis in Plants. Journal of Biological Chemistry 276, 22901-22909.

Gachon, C. M., Langlois-Meurinne, M., Saindrenan, P. (2005) Plant secondary metabolism glycosyltransferases: the emerging functional analysis. Trends in Plant Science 10: 542-549.

Ganeshan, S., Caswell, K. L., Kartha, K. K. and Chibbar, R. N. (2002) Shoot regeneration and proliferation. Transgenic plants and crops. Marcel Dekker, Inc, New York, 69-84.

George, K. W., Thompson, M. G., Kang, A., Baidoo, E., Wang, G., Chan, L. J. G., Adams, P. D., Petzold, C. J., Keasling, J. D. and Soon Lee, T. (2015) Metabolic engineering for the high-yield production of isoprenoid-based C5 alcohols in E. coli. 5, 11128.

Gorst, J. R. (1999) Differentiation and gene expression. In: Plants in Action: Adaptation in Nature, Performance in Cultivation (Atwell, B. J., Kriedemann, P. E. and Turnbull, C. G. N. eds). Macmillan Education AU.

Guleria, P., Masand, S. and Yadav, S. K. (2015) Diversion of carbon flux from gibberellin to steviol biosynthesis by over-expressing SrKA13H induced dwarfism and abnormality in pollen germination and seed set behaviour of transgenic Arabidopsis. J Exp Bot 66, 3907-3916.

Guleria, P., Yadav, S. K. (2013) Agrobacterium mediated transient gene silencing (AMTS) in Stevia rebaudiana: Insights into steviol glycoside biosynthesis pathway. PLoS One 8: e74731.

Hansen, K. S., Kristensen, C., Tattersall, D. B., Jones, P. R., Olsen, C. E., Bak, S., Willer, B. L. (2003) The in vitro substrate regiospecificity of recombinant UGT85B1, the cyanohydrin glucosyltransferase from Sorghum bicolor. Phytochemistry 64: 143-151.

Haseloff, J. et al. (1997). Removal of a cryptic intron and subcellular localization of green fluorescent protein are required to mark transgenic Arabidosis plants brightly. Proc Natl Acad Sci USA 94:2122-2127.

Hellfritsch, C., Brockhoff, A., Stahler, F., Meyerhof, W. and Hofmann, T. (2012) Human Psychometric and Taste Receptor Responses to Steviol Glycosides. Journal of agricultural and food chemistry 60, 6782-6793.

Horsch, R. B., Fry, J. E., Hoffmann, N. L., Eichholtz, D., Rogers, S. G. and Fraley, R. T. (1985) A Simple and General Method for Transferring Genes into Plants. Science 227, 1229-1231.

Humphrey, T. V., Richman, A. S., Menassa, R. and Brandle, J. E. (2006) Spatial Organisation of Four Enzymes from Stevia rebaudiana that are Involved in Steviol Glycoside Synthesis. Plant Molecular Biology 61, 47-62.

Husar, S., Berthiller, F., Fujioka, S., Rozhon, W., Khan, M., Kalaivanan, F., Elias, L., Higgins, G. S., Li, Y., Schuhmacher, R., Krska, R., Seto, H., Vaistij, F. E., Bowles, D., Poppenberger, B. (2011) Overexpression of the UGT73C6 alters brassinosteroid glucoside formation in Arabidopsis thaliana. BMC Plant Biology 11: 51.

Ikeuchi, M., Ogawa, Y., Iwase, A. and Sugimoto, K. (2016) Plant regeneration: cellular origins and molecular mechanisms. Development 143, 1442.

Jones, P., Messner, B., Nakajima, J. I., Schäffner, A. R., Saito, K. (2003) UGT73C6 and UGT78D1, glycosyltransferases involved in flavonol glycoside biosynthesis in Arabidopsis thaliana. Journal of Biological Chemistry 278: 43910-43918.

Karimi, M., Inzé, D. and Depicker, A. (2002) GATEWAY™ vectors for Agrobacterium-mediated plant transformation. Trends in Plant Science 7, 193-195.

Khalil, S. A., Zamir, R. and Ahmad, N. (2014) Selection of suitable propagation method for consistent plantlets production in Stevia rebaudiana (Bertoni). Saudi Journal of Biological Sciences 21, 566-573.

Khan, S. A., Ur Rahman, L., Shanker, K. and Singh, M. (2014) Agrobacterium tumefaciens-mediated transgenic plant and somaclone production through direct and indirect regeneration from leaves in Stevia rebaudiana with their glycoside profile. Protoplasma 251, 661-670.

Kim, M. J., Jin, J., Zheng, J., Wong, L., Chua, N. H. and Jang, I. C. (2015) Comparative Transcriptomics Unravel Biochemical Specialization of Leaf Tissues of Stevia for Diterpenoid Production. Plant Physiology 169, 2462.

Kinghorn, A. D. (2003) Overview. In: Stevia: The Genus Stevia pp. 1-17. CRC Press.

Last, D. I. et al. (1991). pEmu: an improved promoter for gene expression in cereal cells. Theor Appl Genet 81:581-588.

Leclercq, J., Lardet, L., Martin, F., Chapuset, T., Oliver, G. and Montoro, P. (2010) The green fluorescent protein as an efficient selection marker for Agrobacterium tumefaciens-mediated transformation in Hevea brasiliensis (Mull. Arg). Plant Cell Reports 29, 513-522.

Lemus-Mondaca, R., Vega-Gálvez, A., Zura-Bravo, L. and Ah-Hen, K. (2012) Stevia rebaudiana Bertoni, source of a high-potency natural sweetener: A comprehensive review on the biochemical, nutritional and functional aspects. Food Chemistry 132, 1121-1132.

Lichtenthaler, H. K., Wellburn, A. R. (1983) Determinations of total carotenoids and chlorophylls a and b of leaf extracts in different solvents. Biochemical Society Transactions 11: 591

Liu, Z., Yan, J. P., Li, D. K., Luo, Q., Yan, Q., Liu, Z. B., Ye, L. M., Wang, J. M., Li, X. F., Yang, Y. (2015) UDP-Glucosyltransferase 7105, a major glucosyltransferase, mediates abscisic acid homeostasis in Arabidopsis. Plant Physiology 167: 1659-1670.

Lois, L. M., Rodriguez-Concepcion, M., Gallego, F., Campos, N. and Boronat, A. (2000) Carotenoid biosynthesis during tomato fruit development: regulatory role of 1-deoxy-D-xylulose 5-phosphate synthase. The Plant journal : for cell and molecular biology 22, 503-513.

Ma, L. Q., Liu, B. Y., Gao, D. Y., Pang, X. B., Lü, S. Y., Yu, H. S., Wang, H., Yan, F., Li, Z. Q., Li, Y. F., Ye, H. C. (2007) Molecular cloning and overexpression of a novel UDP-glucosyltransferase elevating salidroside levels in Rhodiola sachalinensis. Plant Cell Reports 26: 989-999.

Marutani-Hert, M., Bowman, K. D., McCollum, G. T., Mirkov, T. E., Evens, T. J. and Niedz, R. P. (2012) A Dark Incubation Period Is Important for Agrobacterium-Mediated Transformation of Mature Internode Explants of Sweet Orange, Grapefruit, Citron, and a Citrange Rootstock. PLoS One 7, e47426.

McElroy, D. et al. (1990). Isolation of an efficient actin promoter for use in rice transformation. Plant Cell 2:163-171.

Muñoz-Bertomeu, J., Arrillaga, I., Ros, R. and Segura, J. (2006) Up-Regulation of 1-Deoxy-d-Xylulose-5-Phosphate Synthase Enhances Production of Essential Oils in Transgenic Spike Lavender. Plant Physiology 142, 890.

Nameth, B., Dinka, S. J., Chatfield, S. P., Morris, A., English, J., Lewis, D., Oro, R. and Raizada, M. N. (2013) The shoot regeneration capacity of excised Arabidopsis cotyledons is established during the initial hours after injury and is modulated by a complex genetic network of light signalling. Plant, Cell & Environment 36, 68-86.

Odell, J. T. et al. (1985). Identification of DNA sequences required for activity of the cauliflower mosaic virus 35S promoter. Nature 313:810-812.

Olsson, K., Carlsen, S., Semmler, A., Simón, E., Mikkelsen, M. D., Møller, B. L. (2016) Microbial production of next-generation stevia sweeteners. Microbial Cell Factories 15: 207.

Patel, R. M. and Shah, R. R. (2009) Regeneration of Stevia Plant Through Callus Culture. Indian Journal of Pharmaceutical Sciences 71, 46-50.

Prakash, I., Markosyan, A., Bunders, C. (2014) Development of next generation stevia sweetener: Rebaudioside M. Foods 3: 162-175.

Raina, R., Bh, S. K. and Sharma, Y. (2013) Strategies to improve poor seed germination in Stevia rebaudiana, a low calorie sweetener. Journal of Medicinal Plants Research 7, 1793-1799.

Renault, H., Bassard, J. E., Hamberger, B. and Werck-Reichhart, D. (2014) Cytochrome P450-mediated metabolic engineering: current progress and future challenges. Current Opinion in Plant Biology 19, 27-34.

Richman, A., Swanson, A., Humphrey, T., Chapman, R., McGarvey, B., Pocs, R. and Brandle, J. (2005) Functional genomics uncovers three glucosyltransferases involved in the synthesis of the major sweet glucosides of Stevia rebaudiana. The Plant journal : for cell and molecular biology 41, 56-67.

Richman, A. S., Gijzen, M., Starratt, A. N., Yang, Z. and Brandle, J. E. (1999) Diterpene synthesis in Stevia rebaudiana: recruitment and up-regulation of key enzymes from the gibberellin biosynthetic pathway. The Plant Journal 19, 411-421.

Rodríguez-Concepción, M. and Boronat, A. (2002) Elucidation of the Methylerythritol Phosphate Pathway for Isoprenoid Biosynthesis in Bacteria and Plastids. A Metabolic Milestone Achieved through Genomics. Plant Physiology 130, 1079.

Rogers, S. O. and Bendich, A. J. (1989) Extraction of DNA from plant tissues. In: Plant Molecular Biology Manual (Gelvin, S.B., Schilperoort, R.A. and Verma, D. P. S. eds), pp. 73-83. Dordrecht: Springer Netherlands.

Sahoo, K. K., Tripathi, A. K., Pareek, A., Sopory, S. K. and Singla-Pareek, S. L. (2011) An improved protocol for efficient transformation and regeneration of diverse indica rice cultivars. Plant Methods 7, 49-49.

Singla, R., Jaitak, V. (2016) Synthesis of rebaudioside A from stevioside and their interaction model with hTAS2R4 bitter taste receptor. Phytochemistry 125: 106-111.

Su, Y. H., Liu, Y. B. and Zhang, X. S. (2011) Auxin-Cytokinin Interaction Regulates Meristem Development. Molecular Plant 4, 616-625.

Tai, M. and Stephanopoulos, G. (2013) Engineering the push and pull of lipid biosynthesis in oleaginous yeast Yarrowia lipolytica for biofuel production. Metabolic engineering 15, 1-9.

Tanaka, O. (1997) Improvement of taste of natural sweeteners. In: Pure and Applied Chemistry p. 675.

Thomas, S. G. and Sun, T. P. (2004) Update on Gibberellin Signaling. A Tale of the Tall and the Short. Plant Physiology 135, 668-676.

Totté, N., Charon, L., Rohmer, M., Compernolle, F., Baboeuf, I. and Geuns, J. M. C. (2000) Biosynthesis of the diterpenoid steviol, an ent-kaurene derivative from Stevia rebaudiana Bertoni, via the methylerythritol phosphate pathway. Tetrahedron Letters 41, 6407-6410.

van der Meer, I. M. (2006) Agrobacterium-Mediated Transformation of Petunia Leaf Discs. In: Plant Cell Culture Protocols (Loyola-Vargas, V. M. and Vázquez-Flota, F. eds), pp. 265-272. Totowa, N.J.: Humana Press.

Velten, J. et al. (1984). Isolation of a dual plant promoter fragment from the Ti plasmid of Agrobacterium tumefaciens. EMBO J 3:2723-2730.

Wang, J., Li, S., Xiong, Z. and Wang, Y. (2016). Pathway mining-based integration of critical enzyme parts for de novo biosynthesis of steviolglycosides sweetener in Escherichia coli. Cell Res 26, 258-261.

Zhang, G. et al. (1996). An enhanced green fluorescent protein allows sensitive detection of gene transfer in mammalian cells. Biochem Biophys Res Commun 227:707-711.

1. A method for Agrobacterium-mediated transformation of Stevia plants comprising:
(a) co-culturing leaf explants with Agrobacterium on a solid co-culturing medium which comprises MS mineral salts, MS vitamins, sucrose, acetosyringone (AS) and 2,4-dichlorophenoxyacetic acid (2,4-D) in the dark for a period of time to produce transgenic leaf explants, wherein the Agrobacterium contains a nucleic acid construct to be integrated into the plant genome;
(b) culturing transgenic leaf explants on a solid callus induction medium which comprises MS mineral salts, MS vitamins, sucrose, 6-benzylaminopurine (BA), 3-indoleacetic acid (IAA), a selective agent and an Agrobacterium eradicant in the dark for a period of time to produce transgenic leaf explants with transgenic callus tissue;
(c) culturing the transgenic callus tissue on a solid shoot induction medium which comprises MS mineral salts, MS vitamins, sucrose, BA, IAA, a selective agent and an Agrobacterium eradicant in the dark for a period of time to produce transgenic shoots; and
(d) culturing the transgenic shoots on a solid rooting medium which comprises MS mineral salts, MS vitamins, sucrose and IAA in a light/dark cycle for a period of time to produce transgenic plants.

2. The method of claim 1, wherein the transgenic plants are propagated and maintained in vitro by cutting and transferring apical tissue onto the solid rooting medium every three to four weeks and culturing in a light/dark cycle to produce transgenic plants.

3. The method of claim 1, wherein the concentrations of media components are:
(a) about 3% sucrose, about 0.25 mg/L 2,4-D and about 100 μM AS in the co-culturing medium;
(b) about 3% sucrose, about 1.0 mg/L BA and about 0.5 mg/L IAA in the callus induction medium;
(c) about 3% sucrose, about 1.0 mg/L to about 2mg/L BA and about 0.25 mg/L to about 0.5 mg/L IAA in the shoot induction medium; and
(d) about 3% sucrose and about 0.5 mg/L IAA in the rooting medium;

4. The method of claim 3, wherein the concentration of the components in the shoot induction medium are about 2mg/L BA and about 0.25 mg/L IAA.

5. The method of claim 1, wherein periods of time for the culturing are:
(a) about 2-3 days on the co-culturing medium;
(b) about three weeks to about four weeks, preferably about three weeks on the callus induction medium;
(c) about three weeks to about four weeks, preferably about three weeks on the shoot induction medium; and
(d) about three weeks to about four weeks, preferably about three weeks on the rooting medium.

6. A method for regeneration of Stevia plants comprising:
(a) culturing transgenic leaf explants on a solid callus induction medium which comprises MS mineral salts, MS vitamins, sucrose, 6-benzylaminopurine (BA) and 3-indoleacetic acid (IAA) in the dark for a period of time to produce leaf explants with callus tissue;
(b) culturing the callus tissue on a solid shoot induction medium which comprises MS mineral salts, MS vitamins, sucrose, BA and IAA in the dark for a period of time to produce shoots; and
(c) culturing the shoots on a solid rooting medium which comprises MS mineral salts, MS vitamins, sucrose and IAA in a light/dark cycle for a period of time to produce plants.

7. The method of claim 6, wherein the leaf explants are first co-cultured on a solid co-culturing medium which comprises MS mineral salts, MS vitamins, sucrose and 2,4-dichlorophenoxyacetic acid (2,4-D) in the dark for a period of time to produce co-cultured leaf explants.

8. The method of claim 7, wherein the co-culturing medium for comprises acetosyringone (AS).

9. The method of claim 6, wherein the transgenic plants are propagated and maintained in vitro by cutting and transferring apical tissue onto the solid rooting medium every three to four weeks and culturing in a light/dark cycle to produce transgenic plants.

10. The method of claim 6, wherein the concentrations of media components are:
(a) about 3% sucrose, about 0.25 mg/L 2,4-D and, if present, about 100 μM AS in the co-culturing medium;
(b) about 3% sucrose, about 1.0 mg/L BA and about 0.5 mg/L IAA in the callus induction medium;
(c) about 3% sucrose, about 1.0 mg/L to about 2mg/L BA and about 0.25 mg/L to about 0.5 mg/L IAA in the shoot induction medium; and
(d) about 3% sucrose and about 0.5 mg/L IAA in the rooting medium;

11. The method of claim 10, wherein the concentration of the components in the shoot induction medium are about 2mg/L BA and about 0.25 mg/L IAA.

12. The method of claim 6, wherein periods of time for the culturing are:
(a) about 2-3 days on the co-culturing medium;
(b) about three weeks to about four weeks, preferably about three weeks on the callus induction medium;
(c) about three weeks to about four weeks, preferably about three weeks on the shoot induction medium; and
(d) about three weeks to about four weeks, preferably about three weeks on the rooting medium.

13. A transgenic Stevia plant comprising a polynucleotide selected from the group consisting of:
(a) a polynucleotide encoding SrDXS1 having the amino acid sequence set forth in SEQ ID NO:2;
(b) a polynucleotide encoding SrKAH having the amino acid sequence set forth in SEQ ID NO:4;
(c) a polynucleotide encoding SrUGT76G1 having the amino acid sequence set forth in SEQ ID NO:30;
(d) a polynucleotide encoding SrUGT74G1 having the amino acid sequence set forth in SEQ ID NO:32; and
(f) a polynucleotide encoding SrUGT85C2 having the amino acid sequence set forth in SEQ ID NO:34.

14. The transgenic Stevia plant of claim 13, wherein the transgenic Stevia plant overexpress SrDXS1 and has an enhanced content of steviol glycosides of about 42% to about 54% compared to a wild type Stevia plant.

15. The transgenic Stevia plant of claim 13, wherein the transgenic Stevia plant overexpress SrKAH and has an enhanced content of steviol glycosides of about 67% to about 88% compared to a wild type Stevia plant.

16. A method for producing a transgenic Stevia plant comprising introducing a polynucleotide into a Stevia plant, wherein the polynucleotide is stably integrated into the genome of the transgenic plant and wherein the polynucleotide is selected from the group consisting of:
(a) a polynucleotide encoding SrDXS1 having the amino acid sequence set forth in SEQ ID NO:2;
(b) a polynucleotide encoding SrKAH having the amino acid sequence set forth in SEQ ID NO:4;
(c) a polynucleotide encoding SrUGT76G1 having the amino acid sequence set forth in SEQ ID NO:30;
(d) a polynucleotide encoding SrUGT74G1 having the amino acid sequence set forth in SEQ ID NO:32; and
(f) a polynucleotide encoding SrUGT85C2 having the amino acid sequence set forth in SEQ ID NO:34.

17. The method of claim 16, wherein the transgenic Stevia plant overexpress SrDXS1 and has an enhanced content of steviol glycosides of about 42% to about 54% compared to a wild type Stevia plant.

18. The method of claim 16, wherein the transgenic Stevia plant overexpress SrKAH and has an enhanced content of steviol glycosides of about 67% to about 88% compared to a wild type Stevia plant.

19. The transgenic plant of claim 13, wherein the transgenic plant overexpresses SrUGT76G1 and has an enhanced ratio of rebaudioside A (Reb A) to stevioside of about 207% to about 517% compared to a wild type Stevia plant.

20. The transgenic plant of claim 19, wherein the transgenic plant overexpresses SrUGT76G1 and has an enhanced ratio of rebaudioside C (Reb C) to dulcoside A of about 135% to about 222% compared to a wild type Stevia plant.

21. The transgenic plant of claim 16, wherein the transgenic plant overexpresses SrUGT76G1 and has an enhanced ratio of rebaudioside A (Reb A) to stevioside of about 207% to about 517% compared to a wild type Stevia plant.

22. The transgenic plant of claim 21, wherein the transgenic plant overexpresses SrUGT76G1 and has an enhanced ratio of rebaudioside C (Reb C) to dulcoside A of about 135% to about 222% compared to a wild type Stevia plant.


SEQUENCE LISTING (72 sequences)

SEQ ID NO: 1 - DNA, 2885 bp, Stevia rebaudiana
  CDS (335)..(2479)
gttaatttagataagtgggggatgcaccattatcatgaaatggttgctatccatacaatgtgatttcctattacgtggaccaatcaagaatagctcacgatttcatttcttctttacttgtcttcttacaacatccaccaagttgcttactttcacgaacacaaccatatttaccagtgccaccaccactaccatcacctccgctcaaatcaacaacaatcactattttatcttcaattcccatctgggtctccgtgccatcacacatttccagtttcatcctgcttcttgatccaatttgctcaatcgaacgtgcaaatcgaccctttttccgatggcgatttgtgcctttgcattcccggcacagatgaatcaccggtcgattactaatactccggtgtttcaacattatttaattggaaaggatctgcaacaccatcaatcttcacacaaacccttcagtcagagtaatagattacgtgtggttcaagcaacactgtcggagagaggtgagtatcactcgcagagacctccgactccacttctggacaccatcaattacccaattcacatgaaaaatctctcgattaaggaattgaaacaactagctgatgagctcaggtctgatgtcatctttaacgtttctaaaaccggtggtcatttgggttcgagtcttggtgtggtggagttaacagtggctcttcattacgtgttcaatacgccacaagataagatactttgggatgttggtcatcagtcttacccacataaaatcttgaccggaagaagagaccgaatgcataccattagacagacaaacgggttggcgggtttcactaaacgggctgaaagtgagcatgattgtttcggtacgggtcacagttctaccaccatttctgcagggttaggtatggctgttggaagagatttaaaaggaggaacaaacgatgtaattgcaatcataggcgacggtgcaatgaccgcgggtcaagcttatgaagcaatgaacaacgcgggttatctagattccgacatgatagttatccttaacgacaacaaacaagtctcattaccaactgcaacacttgacgggccaataccgcccgttggtgctttaagcagtgctctgagtcggttacaatctaaccgtccactcagagaactgcgtgaagttgccaaagaagttaccaaacaaattggtgggcccatgcatgaaatcgctgctaaagttgatgaatacgctcgtggtatgattagtgggtcagggtcgaccctgtttgaagaacttgggttgtattatattggacccgttgatggtcatagtattgatgatcttgttgctattcttaaagaggttaagagtacgaaaacaaccggaccggttttgatccatgttattactgagaaaggtagagggtatccttatgccgaaaaagctgctgataaataccatggtgtgacaaagtttgatccggcaacagggaaacaattcaagtcaagtgcaccaactcaatcatacacaacttactttgcagaggcattaatagctgaagctgaggtggacaaaaagattatcggtatccatgctgcaatgggcggtgggaccggtatgaacctgtttcaccgccggttcccgagccggtgttttgatgtcgggattgctgaacaacacgcggttacgtttgcagcgggtttagcgtgcgaaggactcaaaccgttttgtgcaatttactcatcattcctgcaaagaggttatgatcaagtggtgcatgatgtggatttgcagaagttgcctgtgagatttgctatggatcgtgctgggcttgtgggtgctgatgggccgacccattcgggctcgtttgatgtcacttacatggcttgtttgccaaatatggtggttatggctcctgctgatgaggctgaactttttcatatggttgcaaccgcagctgccattgatgatagaccgagttgtttccgttatccgcgtggaaacggggttggtgttatgttgccacctggcaataaaggcgttcctctcgagattggaaaaggtcgaataatgattgaaggcgagcgggttgcacttctcgggtatggaaccgcggttcaaagttgcttggttgcagctggtttagtaaaggaacgaggtttgaacataaccgttgcagacgctcggttttgcaaaccgttggaccagaatctcattcgggcccttgcgaagtcgcatgaggtcttgatcacagttgaagaaggctcaatcggtgggttcgggtcacatgtcgcgcatttcatggcattagatggtcttcttgatggaaaactaaagtggagaccactggttctaccggaccgttacattgaccatggtgcaccggaataccaactagccgaagccgggttgacgccgtcgcatatagctgcaaccgtattcaacgttctcgggcaaacccgagaggctctggaggttatgtcatagaagattcttgtgtgttcactaatgtacacaaatggatatatatatgtatatctgcatagatgtacaatagttgtgatgtgagttgttgctgaacttttaacagtctaaaatagagtcaaccctttgtaaaaagtcaactgaattggattgggtttgtaaagttttagatagtttggtagttaagttgtgataaggaaattgttacgggtcaatgcgaagagacccgtttgaagacccgataatttaaactgaagacgcaataaccagttatctatatatagaaggttttattttctgaatgatttgttagttttagcatttggaaaacataaaaaaatgcctcgacgtgtattggtgtgttagattaattatattgacaacttataatcttatatatcatatcgac

SEQ ID NO: 2 - protein, 714 aa, Stevia rebaudiana
MAICAFAFPAQMNHRSITNTPVFQHYLIGKDLQHHQSSHKPFSQSNRLRVVQATLSERGEYHSQRPPTPLLDTINYPIHMKNLSIKELKQLADELRSDVIFNVSKTGGHLGSSLGVVELTVALHYVFNTPQDKILWDVGHQSYPHKILTGRRDRMHTIRQTNGLAGFTKRAESEHDCFGTGHSSTTISAGLGMAVGRDLKGGTNDVIAIIGDGAMTAGQAYEAMNNAGYLDSDMIVILNDNKQVSLPTATLDGPIPPVGALSSALSRLQSNRPLRELREVAKEVTKQIGGPMHEIAAKVDEYARGMISGSGSTLFEELGLYYIGPVDGHSIDDLVAILKEVKSTKTTGPVLIHVITEKGRGYPYAEKAADKYHGVTKFDPATGKQFKSSAPTQSYTTYFAEALIAEAEVDKKIIGIHAAMGGGTGMNLFHRRFPSRCFDVGIAEQHAVTFAAGLACEGLKPFCAIYSSFLQRGYDQVVHDVDLQKLPVRFAMDRAGLVGADGPTHSGSFDVTYMACLPNMVVMAPADEAELFHMVATAAAIDDRPSCFRYPRGNGVGVMLPPGNKGVPLEIGKGRIMIEGERVALLGYGTAVQSCLVAAGLVKERGLNITVADARFCKPLDQNLIRALAKSHEVLITVEEGSIGGFGSHVAHFMALDGLLDGKLKWRPLVLPDRYIDHGAPEYQLAEAGLTPSHIAATVFNVLGQTREALEVMS

SEQ ID NO: 3 - DNA, 1471 bp, Stevia rebaudiana
  CDS (1)..(1431)
atgattcaagttctaacaccgatccttctcttcctcattttcttcgttttctggaaggtttacaagcaccagaaaaccaaaatcaatcttccaccgggaagcttcggatggccatttctgggcgaaactctggcactcctacgtgcaggttgggactcagagccggagagatttgttcgtgaacggatcaagaaacacggaagtcctctagtgtttaagacgtcgttgtttggcgaccgttttgcggtgttgtgtggacctgccggaaacaagttcctgttctgcaacgagaacaagctggtggcgtcgtggtggccggttccggtgaggaagcttttcggcaagtctctgctcacgattcgtggtgatgaagctaagtggatgaggaagatgttgttatcgtatctcggtcctgatgctttcgcaactcattatgccgtcaccatggacgtcgtcacccgtcggcatatcgacgttcattggcgagggaaggaagaggtgaacgtattccaaaccgttaagttatatgcctttgagcttgcatgtcgtttattcatgaacctagacgacccaaaccacattgcaaaactcggttccttgttcaacattttcttgaaaggcatcattgagcttccaatcgacgtcccagggacacgattttatagctccaaaaaagcagcagcagctatcaggattgaactaaaaaaattgattaaagcaagaaaactggaactgaaagaagggaaggcatcatcttcacaagacctcttatcacatttgcttacatctccagatgaaaatggtatgtttctaaccgaagaagagattgtagacaacatcttgttactactctttgcgggtcatgatacctcggctctttcaatcactttgctcatgaagactcttggcgaacattctgatgtttatgacaaggtgttaaaagagcaactagagatatcgaagacgaaagaagcatgggagtccctgaaatgggaggacatacaaaagatgaaatactcctggagtgttatatgtgaagtcatgagactaaatccacctgttataggaacctatagagaggcccttgtggatattgattatgcgggttataccatccccaaaggatggaagctgcactggagtgctgtatcgacacaaagggacgaggctaactttgaagacgtaacacgttttgacccatcacggtttgaaggcgcaggaccgactccattcacctttgttccgtttggaggggggcctagaatgtgtttagggaaagaatttgctcgattggaagtacttgcgtttcttcacaatattgtcaccaatttcaaatgggacctgttgatacctgatgagaaaatagaatatgatcccatggctaccccagcaaaggggcttccaattcgtcttcatccccatcaagtttgattacttcaagcatgaatcagtgatgtgaaggtaaaccata

SEQ ID NO: 4 - protein, 476 aa, Stevia rebaudiana
MIQVLTPILLFLIFFVFWKVYKHQKTKINLPPGSFGWPFLGETLALLRAGWDSEPERFVRERIKKHGSPLVFKTSLFGDRFAVLCGPAGNKFLFCNENKLVASWWPVPVRKLFGKSLLTIRGDEAKWMRKMLLSYLGPDAFATHYAVTMDVVTRRHIDVHWRGKEEVNVFQTVKLYAFELACRLFMNLDDPNHIAKLGSLFNIFLKGIIELPIDVPGTRFYSSKKAAAAIRIELKKLIKARKLELKEGKASSSQDLLSHLLTSPDENGMFLTEEEIVDNILLLLFAGHDTSALSITLLMKTLGEHSDVYDKVLKEQLEISKTKEAWESLKWEDIQKMKYSWSVICEVMRLNPPVIGTYREALVDIDYAGYTIPKGWKLHWSAVSTQRDEANFEDVTRFDPSRFEGAGPTPFTFVPFGGGPRMCLGKEFARLEVLAFLHNIVTNFKWDLLIPDEKIEYDPMATPAKGLPIRLHPHQV

SEQ ID NO: 5 - DNA, 41 bp, Artificial Sequence
  primer
aaaaagcaggcttcatggcgatttgtgcctttgcattcccg

SEQ ID NO: 6 - DNA, 38 bp, Artificial Sequence
  primer
agaaagctgggtgtgacataacctccagagcctctcgg

SEQ ID NO: 7 - DNA, 42 bp, Artificial Sequence
  primer
aaaaagcaggcttcatggctttatgtggtgctttgaagggtg

SEQ ID NO: 8 - DNA, 45 bp, Artificial Sequence
  primer
agaaagctgggtgtaatacattgacagcatgtagcatctccttgc

SEQ ID NO: 9 - DNA, 45 bp, Artificial Sequence
  primer
aaaaagcaggcttcatgactactgcttctgcacattgttctttgg

SEQ ID NO: 10 - DNA, 42 bp, Artificial Sequence
  primer
agaaagctgggtgacacatcaaaagaagagcttcacgggttc

SEQ ID NO: 11 - DNA, 40 bp, Artificial Sequence
  primer
aaaaagcaggcttcatggcggttgcaggatcgaccatgaa

SEQ ID NO: 12 - DNA, 47 bp, Artificial Sequence
  primer
agaaagctgggtgcattattgatttgtattgaagtgcttctttaggt

SEQ ID NO: 13 - DNA, 39 bp, Artificial Sequence
  primer
aaaaagcaggcttcatgattcaagttctaacaccgatcc

SEQ ID NO: 14 - DNA, 37 bp, Artificial Sequence
  primer
agaaagctgggtgtcaaacttgatggggatgaagacg

SEQ ID NO: 15 - DNA, 38 bp, Artificial Sequence
  primer
aaaaagcaggcttcggagcagctatcaggattgaacta

SEQ ID NO: 16 - DNA, 37 bp, Artificial Sequence; identical to SEQ ID NO: 14
  primer

SEQ ID NO: 17 - DNA, 22 bp, Artificial Sequence
  primer
gcaacactgtcggagagaggtg

SEQ ID NO: 18 - DNA, 24 bp, Artificial Sequence
  primer
ctgttaactccaccacaccaagac

SEQ ID NO: 19 - DNA, 24 bp, Artificial Sequence
  primer
gagcaactagagatatcgaagacg

SEQ ID NO: 20 - DNA, 23 bp, Artificial Sequence
  primer
cactccagtgtagcttccatcct

SEQ ID NO: 21 - DNA, 20 bp, Artificial Sequence
  primer
tcttgatcttgctggtcgtg

SEQ ID NO: 22 - DNA, 20 bp, Artificial Sequence
  primer
gagcaagaacttgaaaccgc

SEQ ID NO: 23 - DNA, 22 bp, Artificial Sequence
  primer
tagagaggcctacgcggcaggt

SEQ ID NO: 24 - DNA, 38 bp, Artificial Sequence; identical to SEQ ID NO: 6
  primer

SEQ ID NO: 25 - DNA, 22 bp, Artificial Sequence
  primer
tagagaggcctacgcggcaggt

SEQ ID NO: 26 - DNA, 37 bp, Artificial Sequence; identical to SEQ ID NO: 14
  primer

SEQ ID NO: 27 - DNA, 28 bp, Artificial Sequence
  primer
atgattgaacaagatggattgcacgcag

SEQ ID NO: 28 - DNA, 28 bp, Artificial Sequence
  primer
tcagaagaactcgtcaagaaggcgatag

SEQ ID NO: 29 - DNA, 1616 bp, Stevia rebaudiana
  CDS (28)..(1404)
cttgcgtgtaaacgtcagtcaaacccaatggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttgtaaataacacgatgattaatcaagcacttggattgcatgctagctgagtagctggtaatttgagttattagaagcaaagactacttggtttaaattaaataaaggatggttgttggttatgtgagctagtttatgttatgttttgtaggctataaaagccttcatatgtttcttattgtttctgtttctaaggtgaaaaaaatgctcgtttttat

SEQ ID NO: 30 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 31 - DNA, 1555 bp, Stevia rebaudiana
  CDS (1)..(1383)
atggcggaacaacaaaagatcaagaaatcaccacacgttctactcatcccattccctttacaaggccatataaaccctttcatccagtttggcaaacgattaatctccaaaggtgtcaaaacaacacttgttaccaccatccacaccttaaactcaaccctaaaccacagtaacaccaccaccacctccatcgaaatccaagcaatttccgatggttgtgatgaaggcggttttatgagtgcaggagaatcatatttggaaacattcaaacaagttgggtctaaatcactagctgacttaatcaagaagcttcaaagtgaaggaaccacaattgatgcaatcatttatgattctatgactgaatgggttttagatgttgcaattgagtttggaatcgatggtggttcgtttttcactcaagcttgtgttgtaaacagcttatattatcatgttcataagggtttgatttctttgccattgggtgaaactgtttcggttcctggatttccagtgcttcaacggtgggagacaccgttaattttgcagaatcatgagcaaatacagagcccttggtctcagatgttgtttggtcagtttgctaatattgatcaagcacgttgggtcttcacaaatagtttttacaagctcgaggaagaggtaatagagtggacgagaaagatatggaacttgaaggtaatcgggccaacacttccatccatgtaccttgacaaacgacttgatgatgataaagataacggatttaatctctacaaagcaaaccatcatgagtgcatgaactggttagacgataagccaaaggaatcagttgtttacgtagcatttggtagcctggtgaaacatggacccgaacaagtggaagaaatcacacgggctttaatagatagtgatgtcaacttcttgtgggttatcaaacataaagaagagggaaagctcccagaaaatctttcggaagtaataaaaaccggaaagggtttgattgtagcatggtgcaaacaattggatgtgttagcacacgaatcagtaggatgctttgttacacattgtgggttcaactcaactcttgaagcaataagtcttggagtccccgttgttgcaatgcctcaattttcggatcaaactacaaatgccaagcttctagatgaaattttgggtgttggagttagagttaaggctgatgagaatgggatagtgagaagaggaaatcttgcgtcatgtattaagatgattatggaggaggaaagaggagtaataatccgaaagaatgcggtaaaatggaaggatttggctaaagtagccgttcatgaaggtggtagctcagacaatgatattgtcgaatttgtaagtgagctaattaaggcttaaatttttgttgctttgtattttatgtgttatggttttttgatttagatgtattcaattaatattgaatcataactaaattcaagattattgtttgtaatattctttgtcctaaaattttgcgacttaaaacctttagtttataaaaagaaattagaaaatactattgcacgga

SEQ ID NO: 32 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 33 - DNA, 1586 bp, Stevia rebaudiana
  CDS (1)..(1446)
atggatgcaatggctacaactgagaagaaaccacacgtcatcttcataccatttccagcacaaagccacattaaagccatgctcaaactagcacaacttctccaccacaaaggactccagataaccttcgtcaacaccgacttcatccacaaccagtttcttgaatcatcgggcccacattgtctagacggtgcaccgggtttccggttcgaaaccattccggatggtgtttctcacagtccggaagcgagcatcccaatcagagaatcactcttgagatccattgaaaccaacttcttggatcgtttcattgatcttgtaaccaaacttccggatcctccgacttgtattatctcagatgggttcttgtcggttttcacaattgacgctgcaaaaaagcttggaattccggtcatgatgtattggacacttgctgcctgtgggttcatgggtttttaccatattcattctctcattgagaaaggatttgcaccacttaaagatgcaagttacttgacaaatgggtatttggacaccgtcattgattgggttccgggaatggaaggcatccgtctcaaggatttcccgctggactggagcactgacctcaatgacaaagttttgatgttcactacggaagctcctcaaaggtcacacaaggtttcacatcatattttccacacgttcgatgagttggagcctagtattataaaaactttgtcattgaggtataatcacatttacaccatcggcccactgcaattacttcttgatcaaatacccgaagagaaaaagcaaactggaattacgagtctccatggatacagtttagtaaaagaagaaccagagtgtttccagtggcttcagtctaaagaaccaaattccgtcgtttatgtaaattttggaagtactacagtaatgtctttagaagacatgacggaatttggttggggacttgctaatagcaaccattatttcctttggatcatccgatcaaacttggtgataggggaaaatgcagttttgccccctgaacttgaggaacatataaagaaaagaggctttattgctagctggtgttcacaagaaaaggtcttgaagcacccttcggttggagggttcttgactcattgtgggtggggatcgaccatcgagagcttgtctgctggggtgccaatgatatgctggccttattcgtgggaccagctgaccaactgtaggtatatatgcaaagaatgggaggttgggctcgagatgggaaccaaagtgaaacgagatgaagtcaagaggcttgtacaagagttgatgggagaaggaggtcacaaaatgaggaacaaggctaaagattggaaagaaaaggctcgcattgcaatagctcctaacggttcatcttctttgaacatagacaaaatggtcaaggaaatcaccgtgctagcaagaaactagttacaaagttgtttcacattgtgctttctatttaagatgtaactttgttctaatttaatattgtctagatgtattgaaccataagtttagttggtctcaggaattgatttttaatgaaataatggtcattaggggtgagt

SEQ ID NO: 34 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 35 - DNA, 22 bp, Artificial Sequence
  primer
gcaacactgtcggagagaggtg

SEQ ID NO: 36 - DNA, 24 bp, Artificial Sequence
  primer
ctgttaactccaccacaccaagac

SEQ ID NO: 37 - DNA, 22 bp, Artificial Sequence
  primer
tcctgaaggtgctttgaggcgt

SEQ ID NO: 38 - DNA, 24 bp, Artificial Sequence
  primer
gacccgtaaagataatgagcttcg

SEQ ID NO: 39 - DNA, 25 bp, Artificial Sequence
  primer
agatgccagagataacatcagtgtg

SEQ ID NO: 40 - DNA, 23 bp, Artificial Sequence
  primer
atgctccaactcgcaacccatca

SEQ ID NO: 41 - DNA, 22 bp, Artificial Sequence
  primer
caggccgaggtgagattgttca

SEQ ID NO: 42 - DNA, 24 bp, Artificial Sequence
  primer
caggcggttccaaatcatttacac

SEQ ID NO: 43 - DNA, 24 bp, Artificial Sequence
  primer
gctgcgaagctcactctgatggtg

SEQ ID NO: 44 - DNA, 24 bp, Artificial Sequence
  primer
cagcttcatgcatcaatctcactg

SEQ ID NO: 45 - DNA, 23 bp, Artificial Sequence
  primer
aggcacacgtttggtggtatctt

SEQ ID NO: 46 - DNA, 24 bp, Artificial Sequence
  primer
gaaagttatgtggtgaagaacagg

SEQ ID NO: 47 - DNA, 24 bp, Artificial Sequence
  primer
catccttggtggtaagcttaacgg

SEQ ID NO: 48 - DNA, 27 bp, Artificial Sequence
  primer
ctactccatatttactcatcatggttc

SEQ ID NO: 49 - DNA, 24 bp, Artificial Sequence
  primer
catgggttcactcatgctccatgt

SEQ ID NO: 50 - DNA, 22 bp, Artificial Sequence
  primer
tgaagctggattcctggatctc

SEQ ID NO: 51 - DNA, 20 bp, Artificial Sequence
  primer
ttccggtgtaaagcggtatc

SEQ ID NO: 52 - DNA, 20 bp, Artificial Sequence
  primer
cattgctttcacgctctcaa

SEQ ID NO: 53 - DNA, 20 bp, Artificial Sequence
  primer
tccggctttctatggttgac

SEQ ID NO: 54 - DNA, 20 bp, Artificial Sequence
  primer
aaccgaaaggctaaagcaca

SEQ ID NO: 55 - DNA, 20 bp, Artificial Sequence
  primer
tcgattaaaaccggagcaac

SEQ ID NO: 56 - DNA, 20 bp, Artificial Sequence
  primer
cccaaaacagcggtcagtat

SEQ ID NO: 57 - DNA, 24 bp, Artificial Sequence
  primer
gagcaactagagatatcgaagacg

SEQ ID NO: 58 - DNA, 23 bp, Artificial Sequence
  primer
cactccagtgtagcttccatcct

SEQ ID NO: 59 - DNA, 28 bp, Artificial Sequence
  primer
gtcattgaggtataatcacatttacacc

SEQ ID NO: 60 - DNA, 24 bp, Artificial Sequence
  primer
tcaccaagtttgatcggatgatcc

SEQ ID NO: 61 - DNA, 25 bp, Artificial Sequence
  primer
gaaatcaccacacgttctactcatc

SEQ ID NO: 62 - DNA, 23 bp, Artificial Sequence
  primer
gaggtggtggtggtgttactgtg

SEQ ID NO: 63 - DNA, 23 bp, Artificial Sequence
  primer
tattcccggtaccatttcaaggc

SEQ ID NO: 64 - DNA, 24 bp, Artificial Sequence
  primer
cggtagattggaaatgcgttcgtc

SEQ ID NO: 65 - DNA, 20 bp, Artificial Sequence
  primer
tcttgatcttgctggtcgtg

SEQ ID NO: 66 - DNA, 20 bp, Artificial Sequence
  primer
gagcaagaacttgaaaccgc

SEQ ID NO: 67 - DNA, 36 bp, Artificial Sequence
  primer
aaaaagcaggcttcatggaaaataaaacggagacca

SEQ ID NO: 68 - DNA, 38 bp, Artificial Sequence
  primer
agaaagctgggtgttacaacgatgaaatgtaagaaact

SEQ ID NO: 69 - DNA, 22 bp, Artificial Sequence
  primer
tagagaggcctacgcggcaggt

SEQ ID NO: 70 - DNA, 24 bp, Artificial Sequence
  primer
gtcatcccttacgtcagtggagat

SEQ ID NO: 71 - DNA, 24 bp, Artificial Sequence
  primer
atctccactgacgtaagggatgac

SEQ ID NO: 72 - DNA, 28 bp, Artificial Sequence
  primer
ttacaacgatgaaatgtaagaaactaga